SEQUENCE LISTING (98 sequences)

SEQ ID NO: 1 - protein, 8 aa, Artificial
  3A8_VH_CDR1
GYSFTGYT

SEQ ID NO: 2 - protein, 8 aa, Artificial
  3A8_VH_CDR2
INPYNGGT

SEQ ID NO: 3 - protein, 12 aa, Artificial
  3A8_VH_CDR3
ARVGGSSWYFDV

SEQ ID NO: 4 - protein, 12 aa, Artificial
  3A8_VL_CDR1
QSLLYSSNQKNY

SEQ ID NO: 5 - skipped

SEQ ID NO: 6 - protein, 10 aa, Artificial
  3A8_VL_CDR3
QQYYSYPTWT

SEQ ID NO: 7 - protein, 119 aa, Artificial
  3A8_VH amino acid
EVQLQQSGPELVKPGASMKISCKASGYSFTGYTMNWVKQSHGKNLEWIGLINPYNGGTSYNQKFKGKATLTVDKSSSTAYMELLSLTSEDSAVYYCARVGGSSWYFDVWGAGTTVTVSS

SEQ ID NO: 8 - protein, 114 aa, Artificial
  3A8_VL amino acid
DIVMTQSPSSLAVSVGEKVIMSCKSSQSLLYSSNQKNYLAWYQQKPGQSPKLLIYWASTRESGVPDRFTGSGSGTDFTLTISSVKAEDLAVYYCQQYYSYPTWTFGGGTKLEIK

SEQ ID NO: 9 - DNA, 357 bp, Artificial
  3A8_VH nucleotide
gaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtactagctacaaccagaagttcaagggcaaggccacattaactgtagacaagtcatccagcacagcctacatggagctcctcagtctgacatctgaggactctgcagtctattactgtgcaagggtgggcggtagtagctggtacttcgatgtctggggcgcagggaccacggtcaccgtctcctca

SEQ ID NO: 10 - DNA, 342 bp, Artificial
  3A8_VL nucleotide
gacattgtgatgacccagtctccatcctccctagctgtgtcagttggagagaaggttattatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctacgtggacgttcggtggaggcaccaagctggaaatcaaa

SEQ ID NO: 11 - protein, 8 aa, Artificial
  4G11_VH_CDR1
GYSFTGYY

SEQ ID NO: 12 - protein, 8 aa, Artificial
  4G11_VH_CDR2
ISCYNGAT

SEQ ID NO: 13 - protein, 10 aa, Artificial
  4G11_VH_CDR3
ARWDRDWFAY

SEQ ID NO: 14 - protein, 6 aa, Artificial
  4G11_VL_CDR1
QDVGIA

SEQ ID NO: 15 - skipped

SEQ ID NO: 16 - protein, 9 aa, Artificial
  4G11_VL_CDR3
QQYSSYPFT

SEQ ID NO: 17 - protein, 117 aa, Artificial
  4G11_VH amino acid
EVQLQQSGPELVKTGDSVKISCKASGYSFTGYYMHWVKQSHGKSLEWIGYISCYNGATSYSQKFKGKATFTVDTSSSTAYMQFNSLTSEDSAVYYCARWDRDWFAYWGQGTLVTVSA

SEQ ID NO: 18 - protein, 107 aa, Artificial
  4G11_VL amino acid
DIVMTQSHKFMSTSVGDRVSITCKASQDVGIAVAWYQQKPGQSPKLLIYWASTRHTGVPDRFTGSGSGTDFTLTISNVQSEDLADYFCQQYSSYPFTFGSGTKLEIK

SEQ ID NO: 19 - DNA, 351 bp, Artificial
  4G11_VH nucleotide
taggtccagctgcaacagtctggacctgagctagtgaagactggggattcagtgaagatatcctgcaaggcttctggttactcattcactggttactacatgcactgggtcaagcagagccatggaaagagccttgagtggattggatatattagttgttacaatggtgctactagctacagccagaagttcaagggcaaggccacatttactgtagacacatcctccagcacagcctacatgcagttcaacagcctgacatctgaagactctgcggtctattactgtgcaagatgggacagggactggtttgcttactggggccaagggactctggtcactgtctctgca

SEQ ID NO: 20 - DNA, 321 bp, Artificial
  4G11_VL nucleotide
tacattgtgatgacccagtctcacaaattcatgtccacatcagtgggagacagggtcagcatcacctgcaaggccagtcaggatgtgggtattgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatccattcacgttcggctcggggacaaagttggaaataaaa

SEQ ID NO: 21 - protein, 10 aa, Artificial
  5A9_VH_CDR1
GFSITSSSYC

SEQ ID NO: 22 - protein, 7 aa, Artificial
  5A9_VH_CDR2
ICYEGSI

SEQ ID NO: 23 - protein, 13 aa, Artificial
  5A9_VH_CDR3
SRENRLLKDAMDY

SEQ ID NO: 24 - protein, 12 aa, Artificial
  5A9_VL_CDR1
QSLLSSRTRKNY

SEQ ID NO: 25 - skipped

SEQ ID NO: 26 - protein, 8 aa, Artificial
  5A9_VL_CDR3
KQSYNLRT

SEQ ID NO: 27 - protein, 121 aa, Artificial
  5A9_VH amino acid
EVQLEESGPAVIKPSQSLSLTCIVSGFSITSSSYCWHWIRQPPGKGLEWMGRICYEGSIYYSPSIKSRFTISRDTSLNKFFIQLSSVTNEDTAMYYCSRENRLLKDAMDYWGQGTSVTVSS

SEQ ID NO: 28 - protein, 112 aa, Artificial
  5A9_VL amino acid
DIVMTQSPSSLAVSAGEKVTMSCKSSQSLLSSRTRKNYLAWYQQKPGQSPKLLIYWASTRESGVPDRFTGSGSGTDFTLTISSVQAEDLAVYYCKQSYNLRTFGGGTKLEIK

SEQ ID NO: 29 - DNA, 363 bp, Artificial
  5A9_VH nucleotide
caggtgcagctggaggagtctggacctgctgtcatcaagccatcacagtcactgtctctcacctgcatagtctctggattctccatcacaagtagtagttattgctggcactggatccgccagcccccaggaaaggggttagagtggatggggcgcatatgttatgaaggttcaatatactatagtccatccatcaaaagccgcttcaccatctccagagacacatctctgaacaaattctttatccagctgagctctgtgacaaatgaggacacagccatgtactactgttccagggaaaaccgcctactgaaggacgctatggactactggggtcaaggaacctcagtcaccgtctcctca

SEQ ID NO: 30 - DNA, 336 bp, Artificial
  5A9_VL nucleotide
aacattgtgatgacccagtctccatcctccctggctgtgtcagcaggagagaaggtcactatgagctgcaaatccagtcagagtctgctcagcagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaaacaatcttataatcttcggacgttcggtggaggcaccaagctggaaatcaaa

SEQ ID NO: 31 - protein, 8 aa, Artificial
  6G5_VH_CDR1
GYSFTGYT

SEQ ID NO: 32 - protein, 8 aa, Artificial
  6G5_VH_CDR2
INPYNGGT

SEQ ID NO: 33 - protein, 12 aa, Artificial
  6G5_VH_CDR3
ARVGGSSWYFDV

SEQ ID NO: 34 - protein, 12 aa, Artificial
  6G5_VL_CDR1
QSLLYSSNQKNY

SEQ ID NO: 35 - skipped

SEQ ID NO: 36 - protein, 10 aa, Artificial
  6G5_VL_CDR3
QQYYTYPTWT

SEQ ID NO: 37 - protein, 119 aa, Artificial; identical to SEQ ID NO: 7
  6G5_VH amino acid

SEQ ID NO: 38 - protein, 114 aa, Artificial
  6G5_VL amino acid
DIVMTQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYTYPTWTFGGGTKLEIK

SEQ ID NO: 39 - DNA, 357 bp, Artificial
  6G5_VH nucleotide
aaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtactagttacaaccagaagttcaagggcaaggccacattaactgtagacaagtcatccagcacagcctacatggagctcctcagtctgacatctgaggactctgcagtctattactgtgcaagggtgggcggtagtagctggtacttcgatgtctggggcgcagggaccacggtcaccgtctcctca

SEQ ID NO: 40 - DNA, 342 bp, Artificial
  6G5_VL nucleotide
tacattgtgatgacccagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatacctatcctacgtggacgttcggtggaggcaccaagctggaaatcaaa

SEQ ID NO: 41 - protein, 8 aa, Artificial
  7C3_VH_CDR1
GYTFSAYW

SEQ ID NO: 42 - protein, 8 aa, Artificial
  7C3_VH_CDR2
ILPGSGST

SEQ ID NO: 43 - protein, 10 aa, Artificial
  7C3_VH_CDR3
ARGDYYAMDY

SEQ ID NO: 44 - protein, 11 aa, Artificial
  7C3_VL_CDR1
QSLLYSNGKTY

SEQ ID NO: 45 - skipped

SEQ ID NO: 46 - protein, 9 aa, Artificial
  7C3_VL_CDR3
VQGTHFPFT

SEQ ID NO: 47 - protein, 117 aa, Artificial
  7C3_VH amino acid
EVQLQQSGAELMRPGASVKISCKATGYTFSAYWIEWVKQRPGHGLEWIGEILPGSGSTKYNEKFKGKATFTADTSSNTAYMQLSSLTSEDSAVYYCARGDYYAMDYWGQGTSVTVSS

SEQ ID NO: 48 - protein, 112 aa, Artificial
  7C3_VL amino acid
DVLMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPFTFGSGTKLEIK

SEQ ID NO: 49 - DNA, 351 bp, Artificial
  7C3_VH nucleotide
taggtccagctgcaacagtctggagctgagctgatgaggcctggggcctcagtgaagatatcctgcaaggctactggctacacattcagtgcctactggatagagtgggtaaagcagaggcctggacatggccttgagtggattggagagattttacctggaagtggtagtactaaatacaatgagaagttcaagggcaaggccacattcactgcagatacatcctccaacacagcctacatgcaactcagcagcctgacatctgaggactctgccgtctattactgtgcaagaggggattactatgctatggactactggggtcaaggaacctcagtcaccgtctcctca

SEQ ID NO: 50 - DNA, 336 bp, Artificial
  7C3_VL nucleotide
aatgttttgatgacccaaactccactcactttgtcggttaccattggacaaccagcctctatctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggtacacattttccattcacgttcggctcggggacaaagttggaaataaaa

SEQ ID NO: 51 - protein, 8 aa, Artificial
  9E8_VH_CDR1
GYSFTGYT

SEQ ID NO: 52 - protein, 8 aa, Artificial
  9E8_VH_CDR2
INPYNGGT

SEQ ID NO: 53 - protein, 12 aa, Artificial
  9E8_VH_CDR3
ARVGGSSWYFDV

SEQ ID NO: 54 - protein, 12 aa, Artificial
  9E8_VL_CDR1
QSLLYSSNQKNY

SEQ ID NO: 55 - skipped

SEQ ID NO: 56 - protein, 10 aa, Artificial
  9E8_VL_CDR3
QQYYSYPTWT

SEQ ID NO: 57 - protein, 119 aa, Artificial; identical to SEQ ID NO: 7
  9E8_VH amino acid

SEQ ID NO: 58 - protein, 114 aa, Artificial; identical to SEQ ID NO: 8
  9E8_VL amino acid

SEQ ID NO: 59 - DNA, 357 bp, Artificial; identical to SEQ ID NO: 9
  9E8_VH nucleotide

SEQ ID NO: 60 - DNA, 342 bp, Artificial; identical to SEQ ID NO: 10
  9E8_VL nucleotide

SEQ ID NO: 61 - protein, 9 aa, Artificial
  9E11_VH_CDR1
GYSITSDYA

SEQ ID NO: 62 - protein, 7 aa, Artificial
  9E11_VH_CDR2
ISYSGST

SEQ ID NO: 63 - protein, 9 aa, Artificial
  9E11_VH_CDR3
ARGAAGFAY

SEQ ID NO: 64 - protein, 6 aa, Artificial
  9E11_VL_CDR1
QTIGTW

SEQ ID NO: 65 - skipped

SEQ ID NO: 66 - protein, 9 aa, Artificial
  9E11_VL_CDR3
QQLYSTPWT

SEQ ID NO: 67 - protein, 116 aa, Artificial
  9E11_VH amino acid
QVQLKESGPGLVKPSQSLSLTCTVTGYSITSDYAWNWIRQFPGNKLEWMGYISYSGSTRYNPSLKSRISITRDTSKNQFFLQLNSVTTEDTATYYCARGAAGFAYWGQGTLVTVSA

SEQ ID NO: 68 - protein, 107 aa, Artificial
  9E11_VL amino acid
DIVMTQSPASQSASLGESVTITCLASQTIGTWLAWYQQKPGKSPQLLIYAATSLADGVPSRFSGSGSGTEFSFKISSLQAEDFVSYYCQQLYSTPWTFGGGTKLEIK

SEQ ID NO: 69 - DNA, 348 bp, Artificial
  9E11_VH nucleotide
gaggtgcagctgaaggagtcgggacctggcctggtgaaaccttcgcagtctctgtccctcacctgcactgtcactggctactcaatcaccagtgattatgcctggaactggatccggcagtttccaggaaacaaactggagtggatgggctacataagctacagtggaagcactaggtacaacccatctctcaaaagtcgaatctctatcactcgagacacatccaagaaccagttcttcctgcagttgaattctgtgactactgaggacacagccacatattactgtgcaagaggggctgcgggctttgcttactggggccaagggactctggtcactgtctctgca

SEQ ID NO: 70 - DNA, 321 bp, Artificial
  9E11_VL nucleotide
tacattgtgatgacccagtctcctgcctcccagtctgcatctctgggagaaagtgtcaccatcacatgcctggcaagtcagaccattggtacatggttagcatggtatcagcagaaaccagggaaatctcctcagctcctgatttatgctgcaaccagcttggcagatggggtcccatcaaggttcagtggtagtggatctggcacagaattttctttcaagatcagcagcctacaggctgaagattttgtaagttattactgtcaacaactttacagtactccgtggacgttcggtggaggcaccaagctggaaatcaaa

SEQ ID NO: 71 - protein, 15 aa, Artificial
  linker amino acid
GGGGSGGGGSGGGGS

SEQ ID NO: 72 - DNA, 45 bp, Artificial
  linker nucleotide
ggcggaggcggcagcggcggaggcggctctggcggcggcgggagc

SEQ ID NO: 73 - protein, 248 aa, Artificial
  3A8 scFv
DIVMTQSPSSLAVSVGEKVIMSCKSSQSLLYSSNQKNYLAWYQQKPGQSPKLLIYWASTRESGVPDRFTGSGSGTDFTLTISSVKAEDLAVYYCQQYYSYPTWTFGGGTKLEIKGGGGSGGGGSGGGGSEVQLQQSGPELVKPGASMKISCKASGYSFTGYTMNWVKQSHGKNLEWIGLINPYNGGTSYNQKFKGKATLTVDKSSSTAYMELLSLTSEDSAVYYCARVGGSSWYFDVWGAGTTVTVSS

SEQ ID NO: 74 - DNA, 744 bp, Artificial
  3A8 scFv
gacattgtgatgacccagtctccatcctccctagctgtgtcagttggagagaaggttattatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctacgtggacgttcggtggaggcaccaagctggaaatcaaaggcggaggcggcagcggcggaggcggctctggcggcggcgggagcgaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtactagctacaaccagaagttcaagggcaaggccacattaactgtagacaagtcatccagcacagcctacatggagctcctcagtctgacatctgaggactctgcagtctattactgtgcaagggtgggcggtagtagctggtacttcgatgtctggggcgcagggaccacggtcaccgtctcctca

SEQ ID NO: 75 - protein, 239 aa, Artificial
  4G11 scFv
DIVMTQSHKFMSTSVGDRVSITCKASQDVGIAVAWYQQKPGQSPKLLIYWASTRHTGVPDRFTGSGSGTDFTLTISNVQSEDLADYFCQQYSSYPFTFGSGTKLEIKGGGGSGGGGSGGGGSEVQLQQSGPELVKTGDSVKISCKASGYSFTGYYMHWVKQSHGKSLEWIGYISCYNGATSYSQKFKGKATFTVDTSSSTAYMQFNSLTSEDSAVYYCARWDRDWFAYWGQGTLVTVSA

SEQ ID NO: 76 - DNA, 717 bp, Artificial
  4G11 scFv
tacattgtgatgacccagtctcacaaattcatgtccacatcagtgggagacagggtcagcatcacctgcaaggccagtcaggatgtgggtattgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatccattcacgttcggctcggggacaaagttggaaataaaaggcggaggcggcagcggcggaggcggctctggcggcggcgggagctaggtccagctgcaacagtctggacctgagctagtgaagactggggattcagtgaagatatcctgcaaggcttctggttactcattcactggttactacatgcactgggtcaagcagagccatggaaagagccttgagtggattggatatattagttgttacaatggtgctactagctacagccagaagttcaagggcaaggccacatttactgtagacacatcctccagcacagcctacatgcagttcaacagcctgacatctgaagactctgcggtctattactgtgcaagatgggacagggactggtttgcttactggggccaagggactctggtcactgtctctgca

SEQ ID NO: 77 - protein, 248 aa, Artificial
  5A9 scFv
DIVMTQSPSSLAVSAGEKVTMSCKSSQSLLSSRTRKNYLAWYQQKPGQSPKLLIYWASTRESGVPDRFTGSGSGTDFTLTISSVQAEDLAVYYCKQSYNLRTFGGGTKLEIKGGGGSGGGGSGGGGSEVQLEESGPAVIKPSQSLSLTCIVSGFSITSSSYCWHWIRQPPGKGLEWMGRICYEGSIYYSPSIKSRFTISRDTSLNKFFIQLSSVTNEDTAMYYCSRENRLLKDAMDYWGQGTSVTVSS

SEQ ID NO: 78 - DNA, 744 bp, Artificial
  5A9 scFv
aacattgtgatgacccagtctccatcctccctggctgtgtcagcaggagagaaggtcactatgagctgcaaatccagtcagagtctgctcagcagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaaacaatcttataatcttcggacgttcggtggaggcaccaagctggaaatcaaaggcggaggcggcagcggcggaggcggctctggcggcggcgggagccaggtgcagctggaggagtctggacctgctgtcatcaagccatcacagtcactgtctctcacctgcatagtctctggattctccatcacaagtagtagttattgctggcactggatccgccagcccccaggaaaggggttagagtggatggggcgcatatgttatgaaggttcaatatactatagtccatccatcaaaagccgcttcaccatctccagagacacatctctgaacaaattctttatccagctgagctctgtgacaaatgaggacacagccatgtactactgttccagggaaaaccgcctactgaaggacgctatggactactggggtcaaggaacctcagtcaccgtctcctca

SEQ ID NO: 79 - protein, 248 aa, Artificial
  6G5 scFv
DIVMTQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYTYPTWTFGGGTKLEIKGGGGSGGGGSGGGGSEVQLQQSGPELVKPGASMKISCKASGYSFTGYTMNWVKQSHGKNLEWIGLINPYNGGTSYNQKFKGKATLTVDKSSSTAYMELLSLTSEDSAVYYCARVGGSSWYFDVWGAGTTVTVSS

SEQ ID NO: 80 - DNA, 744 bp, Artificial
  6G5 scFv
tacattgtgatgacccagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatacctatcctacgtggacgttcggtggaggcaccaagctggaaatcaaaggcggaggcggcagcggcggaggcggctctggcggcggcgggagcaaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtactagttacaaccagaagttcaagggcaaggccacattaactgtagacaagtcatccagcacagcctacatggagctcctcagtctgacatctgaggactctgcagtctattactgtgcaagggtgggcggtagtagctggtacttcgatgtctggggcgcagggaccacggtcaccgtctcctca

SEQ ID NO: 81 - protein, 244 aa, Artificial
  7C3 scFv
DVLMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPFTFGSGTKLEIKGGGGSGGGGSGGGGSEVQLQQSGAELMRPGASVKISCKATGYTFSAYWIEWVKQRPGHGLEWIGEILPGSGSTKYNEKFKGKATFTADTSSNTAYMQLSSLTSEDSAVYYCARGDYYAMDYWGQGTSVTVSS

SEQ ID NO: 82 - DNA, 732 bp, Artificial
  7C3 scFv
aatgttttgatgacccaaactccactcactttgtcggttaccattggacaaccagcctctatctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggtacacattttccattcacgttcggctcggggacaaagttggaaataaaaggcggaggcggcagcggcggaggcggctctggcggcggcgggagctaggtccagctgcaacagtctggagctgagctgatgaggcctggggcctcagtgaagatatcctgcaaggctactggctacacattcagtgcctactggatagagtgggtaaagcagaggcctggacatggccttgagtggattggagagattttacctggaagtggtagtactaaatacaatgagaagttcaagggcaaggccacattcactgcagatacatcctccaacacagcctacatgcaactcagcagcctgacatctgaggactctgccgtctattactgtgcaagaggggattactatgctatggactactggggtcaaggaacctcagtcaccgtctcctca

SEQ ID NO: 83 - protein, 248 aa, Artificial; identical to SEQ ID NO: 73
  9E8 scFv

SEQ ID NO: 84 - DNA, 744 bp, Artificial; identical to SEQ ID NO: 74
  9E8 scFv

SEQ ID NO: 85 - protein, 177 aa, Artificial
  9E11 scFv
DIVMTQSPASQSASLGESVTITCLASQTIGTWLAWYQQKPGKSPQLLIYAATSLADGVPSRFSGSGSGTEFSFKISSLQAEDFVSYYCQQLYSTPWTFGGGTKLEIKGGGGSGGGGSGGGGSQVQLKESGPGLVKPSQSLSLTCTVTGYSITSDYAWNWIRQFPGNKLEWMGYISYS

SEQ ID NO: 86 - DNA, 713 bp, Artificial
  9E11 scFv
tacattgtgatgacccagtctcctgcctcccagtctgcatctctgggagaaagtgtcaccatcacatgcctggcaagtcagaccattggtacatggttagcatggtatcagcagaaaccagggaaatctcctcagctcctgatttatgctgcaaccagcttggcagatggggtcccatcaaggttcagtggtagtggatctggcacagaattttctttcaagatcagcagcctacaggctgaagattttgtaagttattactgtcaacaactttacagtactccgtggacgttcggtggaggcaccaagctggaaatcaaaggcggaggcggcagcggcggaggcggctctggcggcggcgggagcgaggtgcagctgaaggagtcgggacctggcctggtgaaaccttcgcagtctctgtccctcacctgcactgtcactggctactcaatcaccagtgattatgcctggaactggatccggcagtttccaggaaacaaactggagtggatgggctacataagctacagtggaagcactaggtacaacccatctctcaaaagtcgaatctctatcactcgagacacatccaagaaccagttcttcctgcagttgaattctgtgactactgaggacacagccacatattactgtgcaagaggggctgcgggctttgcttactggggccaagggactctggtcactgtctctgc

SEQ ID NO: 87 - DNA, 1178 bp, Artificial
  EF1 promoter
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 88 - DNA, 63 bp, Artificial
  signal peptide
atggccttaccagtgaccgccttgctcctgccgctggccttgctgctccacgccgccaggccg

SEQ ID NO: 89 - DNA, 135 bp, Artificial
  CD8 Hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 90 - DNA, 72 bp, Artificial
  TM(transmembrane domain)
atctacatctgggcgcccttggccgggacttgtggggtccttctcctgtcactggttatcaccctttactgc

SEQ ID NO: 91 - DNA, 126 bp, Artificial
  4-1BB
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 92 - DNA, 336 bp, Artificial
  CD3 zeta
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 93 - DNA, 591 bp, Artificial
  WPRE
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 94 - protein, 21 aa, Artificial
  signal peptide
MALPVTALLLPLALLLHAARP

SEQ ID NO: 95 - protein, 45 aa, Artificial
  CD8 Hinge
TTTPAPRPPTPAPTIASQPLSLRPEACRPAAGGAVHTRGLDFACD

SEQ ID NO: 96 - protein, 24 aa, Artificial
  TM(transmembrane domain)
IYIWAPLAGTCGVLLLSLVITLYC

SEQ ID NO: 97 - protein, 42 aa, Artificial
  4-1BB
KRGRKKLLYIFKQPFMRPVQTTQEEDGCSCRFPEEEEGGCEL

SEQ ID NO: 98 - protein, 112 aa, Artificial
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0129178 A1
(43) Pub. Date: Apr. 24, 2025

(54) ANTIBODY SPECIFIC FOR MESOTHELIN AND USES THEREOF

(71) Applicant: INNOBATION BIO CO., LTD., Seoul (KR)

(72) Inventors: Seung Ku KIM, Yongin-si (KR); Ki Tae KIM, Seoul (KR)

(73) Assignee: INNOBATION BIO CO., LTD., Seoul (KR)

(21) Appl. No.: 18/575,494

(22) PCT Filed: May 30, 2022
(86) PCT No.: PCT/KR2022/007656

§ 371 Date: Dec. 29, 2023

(30) Foreign Application Priority Data
Jun. 30, 2021 (KR) 10-2021-0085569

(51) Int. Cl. C07K 16/30; A61K 40/11; A61K 40/31; A61K 40/42; A61P 35/00; C07K 14/725; C12N 5/0783

(52) U.S. Cl. CPC C07K 16/30; A61K 40/11; A61K 40/31; A61K 40/4255; A61P 35/00; C07K 14/7051; C12N 5/0636; C07K 2317/53; C07K 2319/02; C07K 2319/03; C12N 2510/00

(57) ABSTRACT

The present invention relates to mesothelin-specific antibody and uses thereof, and more particularly to antibody that specifically bind to mesothelin, chimeric antigen receptors comprising the antibody, CAR-T cells expressing the chimeric antigen receptors, and a pharmaceutical composition for the prevention or treatment of mesothelin-expressing cancers or tumors comprising the antibody.

Seven antibodies specific to mesothelin (3A8, 4G11, 5A9, 6G5, 7C3, 9E8, and 9E11) selected in the present invention were found to specifically bound to mesothelin antigen and enable the preparation of chimeric antigen receptor (CAR) and CAR-T cells (MSLN-CAR-T cells) targeting mesothelin. Furthermore, we have confirmed that the MSLN-CAR-T cells produced by the present invention activate MSLN-CAR-T cells in the presence of mesothelin antigen and effectively killed cells overexpressing mesothelin, so that the antibodies specific for mesothelin and the chimeric antigen receptors and CAR-T cells produced using the same can be applied to the prevention or treatment of cancer or tumors in which mesothelin is overexpressed.

### CROSS-REFERENCE TO RELATED APPLICATION(S)

This application is a 35 U.S.C. 371 National Phase Entry Application from PCT/KR2022/007656 filed on May 30, 2022, which claims priority to and the benefit of Korean Patent Application No. 10-2021-0085569, filed on Jun. 30, 2021, all of the disclosures of which are incorporated herein by reference in their entirety.

The “Sequence Listing” submitted electronically concurrently herewith pursuant 37 C.F.R. § 1.821 in computer readable form (CRF) via EFS-Web as file name POPC214316PCTUS.ST25.txt is incorporated herein by reference. The electronic copy of the Sequence Listing was created on Nov. 25, 2024, and the size on disk is 61,440 bytes.

### TECHNICAL FIELD

The present invention relates to an antibody specific to mesothelin and uses thereof, and more particularly, to an antibody that specifically binds to mesothelin, a chimeric antigen receptor comprising the antibody, a CAR-T cell expressing the chimeric antigen receptor, and a pharmaceutical composition for the prevention or treatment of mesothelin-expressing cancers or tumors comprising the same.

### BACKGROUND

Mesothelin (MSLN) is a 69-71 kDa precursor polypeptide, a glycoprotein, which is expressed on the cell surface to help cells adhere to each other and transmit signals. Although it shows low expression in general tissues, overexpression has been confirmed in several types of solid cancer, including mesothelioma, pancreatic cancer, and ovarian cancer, and anti-cancer target research targeting mesothelin is in progress.

Antibody-based targeted therapies are being developed for lung, ovarian, and pancreatic cancers that express mesothelin (Korean Public Patent No. 10-2017-0036503; Chang, K, et al, Int J Cancer, 50(3):373, 1992), and in particular, chimeric antigen receptor and CAR-T cells using mesothelin-specific antibodies are being actively researched (Korean Published Patent No. 10-2070016; Zhiwei Zhang, et al., Cell Death & Disease, 10:479, 2019).

### SUMMARY OF THE INVENTION

Therefore, in the present invention, in order to develop an antibody that binds more specifically to mesothelin, seven novel antibodies (3A8, 4G11, 5A9, 6G5, 7C3, 9E8, and 9E11) were established by screening antibodies that bind to mesothelin, and it was confirmed that 7 types of antibodies selected in the present invention specifically bound to the mesothelin antigen. In addition, chimeric antigen receptors and CAR-T cells targeting mesothelin were prepared using the mesothelin-specific antibody of the present invention, and it was confirmed that MSLN-CAR-T cells effectively killed mesothelin-overexpressing cells, and the present invention was completed.

Accordingly, an object of the present invention is to provide an antibody that specifically binds to mesothelin.

Another object of the present invention is to provide a polynucleotide encoding the antibody, a vector expressing the antibody, and a recombinant cell transformed with the vector.

Another object of the present invention is to provide a chimeric antigen receptor comprising the antibody that specifically binds to mesothelin, a polynucleotide encoding the chimeric antigen receptor, a vector comprising the polynucleotide, and an immune effector cell expressing the chimeric antigen receptor or vector.

Another object of the present invention is to provide a pharmaceutical composition for the prevention or treatment of cancer or tumors comprising the antibody that specifically binds to mesothelin or the immune effector cell expressing chimeric antigen receptor comprising the antibody.

In order to achieve the above purpose, the present invention is to provide an antibody specifically binding to mesothelin or a fragment thereof, comprising:
(1) a heavy chain variable region including a CDR1 region represented by an amino acid of SEQ ID NO: 1, a CDR2 region represented by an amino acid of SEQ ID NO: 2 and a CDR3 region represented by an amino acid of SEQ ID NO: 3, and a light chain variable region including a CDR1 region represented by an amino acid of SEQ ID NO: 4, a CDR2 region represented by an amino acid sequence WAS and a CDR3 region represented by an amino acid of SEQ ID NO: 6;
(2) a heavy chain variable region including a CDR1 region represented by an amino acid of SEQ ID NO: 11, a CDR2 region represented by an amino acid of SEQ ID NO: 12 and a CDR3 region represented by an amino acid of SEQ ID NO: 13, and a light chain variable region including a CDR1 region represented by an amino acid of SEQ ID NO: 14, a CDR2 region with an amino acid sequence of WAS and a CDR3 region represented by an amino acid of SEQ ID NO: 16;
(3) a heavy chain variable region including a CDR1 region represented by an amino acid of SEQ ID NO: 21, a CDR2 region represented by an amino acid of SEQ ID NO: 22 and a CDR3 region represented by an amino acid of SEQ ID NO: 23, and a light chain variable region including a CDR1 region represented by an amino acid of SEQ ID NO: 24, a CDR2 region with an amino acid sequence: WAS and a CDR3 region represented by an amino acid of SEQ ID NO: 26;
(4) a heavy chain variable region including a CDR1 region represented by an amino acid of SEQ ID NO: 31, a CDR2 region represented by an amino acid of SEQ ID NO: 32 and a CDR3 region represented by an amino acid of SEQ ID NO: 33, and a light chain variable region including a CDR1 region represented by an amino acid of SEQ ID NO: 34, a CDR2 region with an amino acid sequence of WAS and a CDR3 region represented by an amino acid of SEQ ID NO: 36;
(5) a heavy chain variable region including a CDR1 region represented by an amino acid of SEQ ID NO: 41, a CDR2 region represented by an amino acid of SEQ ID NO: 42 and a CDR3 region represented by an amino acid of SEQ ID NO: 43, and a light chain variable region including a CDR1 region represented by an amino acid of SEQ ID NO: 44, a CDR2 region with an amino acid sequence of LVS and a CDR3 region represented by an amino acid of SEQ ID NO: 46;
(6) a heavy chain variable region including a CDR1 region represented by an amino acid of SEQ ID NO: 51, a CDR2 region represented by an amino acid of SEQ ID NO: 52 and a CDR3 region represented by an amino acid of SEQ ID NO: 53, and a light chain variable region including a CDR1 region represented by an amino acid of SEQ ID NO: 54, a CDR2 region with an amino acid sequence of WAS and a CDR3 region represented by an amino acid of SEQ ID NO: 56; or
(7) a heavy chain variable region including a CDR1 region represented by an amino acid of SEQ ID NO: 61, a CDR2 region represented by an amino acid of SEQ ID NO: 62 and a CDR3 region represented by an amino acid of SEQ ID NO: 63, and a light chain variable region including a CDR1 region represented by an amino acid of SEQ ID NO: 64, a CDR2 region with an amino acid sequence of AAT and a CDR3 region represented by an amino acid of SEQ ID NO: 66.

In a preferred embodiment of the present invention, the antibody may be a monoclonal antibody, preferably a single-chain variable fragment (scFv).

In another preferred embodiment of the present invention,
the (1) antibody may comprise a heavy chain variable region represented by an amino acid of SEQ ID NO: 7 and a light chain variable region represented by an amino acid of SEQ ID NO: 8;
the (2) antibody may comprise a heavy chain variable region represented by an amino acid of SEQ ID NO: 17 and a light chain variable region represented by an amino acid of SEQ ID NO: 18;
the (3) antibody may comprise a heavy chain variable region represented by an amino acid of SEQ ID NO: 27 and a light chain variable region represented by an amino acid of SEQ ID NO: 28;
the (4) antibody may comprise a heavy chain variable region represented by an amino acid of SEQ ID NO: 37 and a light chain variable region represented by an amino acid of SEQ ID NO: 38;
the (5) antibody may comprise a heavy chain variable region represented by an amino acid of SEQ ID NO: 47 and a light chain variable region represented by an amino acid of SEQ ID NO: 48;
the (6) antibody may comprise a heavy chain variable region represented by an amino acid of SEQ ID NO: 57 and a light chain variable region represented by an amino acid of SEQ ID NO: 58; or
the (7) antibody may comprise a heavy chain variable region represented by an amino acid of SEQ ID NO: 67 and a light chain variable region represented by an amino acid of SEQ ID NO: 68.

To achieve another object, the present invention provides a polynucleotide encoding the antibody that specifically binds to mesothelin.

In addition, the present invention provides a vector comprising a polynucleotide encoding the antibody that specifically binds to mesothelin.

In addition, the present invention provides a recombinant cell transformed with the vector that produces the antibody or a fragment thereof that specifically binds to mesothelin.

In order to achieve another object, the present invention provides a chimeric antigen receptor (CAR) comprising: a mesothelin-binding domain; a transmembrane domain; a costimulatory domain; and an intracellular signal transduction domain,
wherein the mesothelin-binding domain may be selected from the antibody specifically binding to mesothelin or a fragment thereof.

The mesothelin-binding domain may be selected from the antibody or a fragment thereof capable of specifically binding to mesothelin of the present invention.

In a preferred embodiment of the present invention, the transmembrane domain may be derived from a protein selected from the group consisting of CD8a, CD4, CD28, CD137, CD80, CD86, CD152 and PD1.

In another preferred embodiment of the present invention, the costimulatory domain may be derived from a protein selected from the group consisting of CD28, 4-1BB, OX-40 and ICOS, and the signaling domain may be derived from CD3ζ.

In another preferred embodiment of the present invention, a hinge region located between the C terminus of the mesothelin-binding domain and the N terminus of the transmembrane domain may be further included, wherein the hinge region may be derived from CD8a.

In order to achieve another object, the present invention provides a polynucleotide encoding the chimeric antigen receptor (CAR).

In addition, the present invention provides a vector comprising a polynucleotide encoding a chimeric antigen receptor (CAR).

In a preferred embodiment of the present invention, the vector may be a plasmid, a retroviral vector, or a lentiviral vector.

In addition, the present invention provides an immune effector cell expressing the chimeric antigen receptor (CAR) comprising the polynucleotide encoding the chimeric antigen receptor.

In a preferred embodiment of the present invention, the immune effector cell may be a T cell.

In order to achieve another object, the present invention provides a pharmaceutical composition for use in preventing or treating a cancer or tumor, comprising the antibody specifically binding to mesothelin or the fragment thereof, or the immune effector cell expressing a chimeric antigen receptor targeting mesothelin.

In the present invention, the cancer or tumor may be a cancer or tumor in which mesothelin is overexpressed relative to normal cells.

In the present invention, the cancer or tumor may be selected from the group consisting of squamous cell carcinoma, small cell lung cancer, non-small cell lung cancer, adenocarcinoma of the lung, squamous cell carcinoma of the lung, mesothelioma, peritoneal cancer, hepatocellular carcinoma, gastrointestinal cancer, pancreatic cancer, glioma, cervical cancer, ovarian cancer, liver cancer, bladder cancer, hepatocellular carcinoma, breast cancer, colon cancer, colorectal cancer, endometrial or uterine carcinoma, salivary gland carcinoma, kidney cancer, liver cancer, prostate cancer, vulvar cancer, thyroid cancer, liver carcinoma, leukemia and other lymphoproliferative disorders, and various types of head and neck cancer.

In the present invention, antibodies that more specifically bound to mesothelin were screened to establish seven novel antibodies (3A8, 4G11, 5A9, 6G5, 7C3, 9E8, and 9E11), and it was confirmed that the novel antibodies specifically bound to mesothelin antigen.

In addition, it was confirmed that chimeric antigen receptors (CARs) and MSLN-CAR-T cells prepared in the present invention effectively recognized the mesothelin antigen, induced activation of the CAR-T cells, and effectively killed cells overexpressing mesothelin, thus, the mesothelin-specific antibody of the present invention, the chimeric antigen receptor prepared using the same and CAR-T cell(s) can be applied to the prevention or treatment of cancers or tumors in which mesothelin is overexpressed.

### BRIEF DESCRIPTION OF THE DRAWINGS

FIG. 1 shows flow cytometry data confirming the binding of 3A8, 4G11, 5A9, 6G5, 7C3, 9E8, and 9E11 antibodies selected from the present invention to mesothelioma cells (NCI-H2052) overexpressing mesothelin.

FIG. 2 is a schematic representation of a chimeric antigen receptor targeting mesothelin (MSLN-CAR).

FIG. 3 is a schematic illustrating a method of producing MSLN-CAR expressing cells using a lentivirus expressing MSLN-CAR.

FIG. 4 is a schematic illustrating (A) a method for transfecting a HEK293 cell line with an MSLN-CAR expressing lentivirus and (B) a method for determining the binding capacity of the transduced HEK293 cells to mesothelin peptide.

FIG. 5A and FIG. 5B are data confirming the level of MSLN-CAR expression in HEK293FT cells transduced with a lentivirus expressing MSLN-CAR.

FIG. 6 is a schematic illustrating a method of producing MSLN-CART cells using a lentivirus expressing MSLN-CAR.

FIG. 7 is a schematic illustrating (A) a method for producing MSLN-CART cells using peripheral blood mononuclear cells (PBMCs) and (B) a method for testing the binding capacity of the produced MSLN-CART cells to mesothelin peptide.

FIGS. 8A through 8D are data confirming the binding capacity of MSLN-CAR-T cells prepared using 3A8, 4G11, 6G5, and 7C3 antibodies, respectively, to mesothelin peptides.

FIGS. 9A to 9D are data confirming the extent of IFNγ expression by MSLN-CAR-T cells in the presence of target cells to confirm the activation of MSLN-CAR-T cells prepared using 3A8, 4G11, 6G5, and 7C3 antibodies, respectively.

FIG. 10 is data confirming the killing effect of target cells by MSLN-CAR-T cells prepared using 3A8, 4G11, 6G5, and 7C3 antibodies, respectively.

### DETAILED DESCRIPTION

Hereinafter, the present invention is described in detail.

Antibodies that Specifically Bind to Mesothelin

In one aspect, the present invention relates to an antibody specifically binding to mesothelin or a fragment thereof, comprising:
(1) a heavy chain variable region including a CDR1 region represented by an amino acid of SEQ ID NO: 1, a CDR2 region represented by an amino acid of SEQ ID NO: 2 and a CDR3 region represented by an amino acid of SEQ ID NO: 3, and a light chain variable region including a CDR1 region represented by an amino acid of SEQ ID NO: 4, a CDR2 region with an amino acid sequence: WAS and a CDR3 region represented by an amino acid of SEQ ID NO: 6;
(2) a heavy chain variable region including a CDR1 region represented by an amino acid of SEQ ID NO: 11, a CDR2 region represented by an amino acid of SEQ ID NO: 12 and a CDR3 region represented by an amino acid of SEQ ID NO: 13, and a light chain variable region including a CDR1 region represented by an amino acid of SEQ ID NO: 14, a CDR2 region with an amino acid sequence of WAS and a CDR3 region represented by an amino acid of SEQ ID NO: 16;
(3) a heavy chain variable region including a CDR1 region represented by an amino acid of SEQ ID NO: 21, a CDR2 region represented by an amino acid of SEQ ID NO: 22 and a CDR3 region represented by an amino acid of SEQ ID NO: 23, and a light chain variable region including a CDR1 region represented by an amino acid of SEQ ID NO: 24, a CDR2 region with an amino acid sequence: WAS and a CDR3 region represented by an amino acid of SEQ ID NO: 26;
(4) a heavy chain variable region including a CDR1 region represented by an amino acid of SEQ ID NO: 31, a CDR2 region represented by an amino acid of SEQ ID NO: 32 and a CDR3 region represented by an amino acid of SEQ ID NO: 33, and a light chain variable region including a CDR1 region represented by an amino acid of SEQ ID NO: 34, a CDR2 region with an amino acid sequence of WAS and a CDR3 region represented by an amino acid of SEQ ID NO: 36;
(5) a heavy chain variable region including a CDR1 region represented by an amino acid of SEQ ID NO: 41, a CDR2 region represented by an amino acid of SEQ ID NO: 42 and a CDR3 region represented by an amino acid of SEQ ID NO: 43, and a light chain variable region including a CDR1 region represented by an amino acid of SEQ ID NO: 44, a CDR2 region with an amino acid sequence of LVS and a CDR3 region represented by an amino acid of SEQ ID NO: 46;
(6) a heavy chain variable region including a CDR1 region represented by an amino acid of SEQ ID NO: 51, a CDR2 region represented by an amino acid of SEQ ID NO: 52 and a CDR3 region represented by an amino acid of SEQ ID NO: 53, and a light chain variable region including a CDR1 region represented by an amino acid of SEQ ID NO: 54, a CDR2 region with an amino acid sequence of WAS and a CDR3 region represented by an amino acid of SEQ ID NO: 56; or
(7) a heavy chain variable region including a CDR1 region represented by an amino acid of SEQ ID NO: 61, a CDR2 region represented by an amino acid of SEQ ID NO: 62 and a CDR3 region represented by an amino acid of SEQ ID NO: 63, and a light chain variable region including a CDR1 region represented by an amino acid of SEQ ID NO: 64, a CDR2 region with an amino acid sequence of AAT and a CDR3 region represented by an amino acid of SEQ ID NO: 66.

In the present invention, the antibody may be a monoclonal antibody. In the present invention, the term “monoclonal antibody” may be an antibody produced by a single antibody-forming cell, with a uniform primary structure (amino acid sequence). It recognizes only one antigenic determinant and is generally produced by culturing a hybridoma cell in which cancer cells and an antibody-producing cell are fused, and it can also be produced using other recombinant protein-expressing host cells using a known antibody gene sequence. Furthermore, the antibody may be humanized to exclude the CDR portion as needed.

As used herein, the term “CDR (complementarity determining region)”, refers to a non-contiguous antigen binding site found within the variable region of both heavy and light chain polypeptides.

In the present invention, the term “antibody” can be used not only in a complete form having two full-length light chains and two full-length heavy chains, but also fragments of antibody molecule. A fragment of antibody molecule means a fragment having at least a peptide tag (epitope) binding function, and includes scFv, Fab, F(ab′), F(ab′)2, a single domain, etc.

Among antibody fragments, Fab has a structure having variable regions of light and heavy chains, a constant region of light chain and the first constant region of heavy chain (CH1), and has one antigen-binding site. Fab′ differs from Fab in that it has a hinge region comprising one or more cysteine residues at the C terminus of the heavy chain CH1 domain. F(ab′)₂ antibody is produced by forming a disulfide bond with a cysteine residue in the hinge region of Fab′. Fv is a minimal antibody fragment having only a heavy chain variable region and a heavy chain variable region, a double-chain Fv (dsFv) has a heavy chain variable region and a light chain variable region linked by a disulfide bond, and a short-chain Fv (scFv) has a heavy chain variable region and a light chain variable region linked by a covalent bond, usually through a peptide linker. These antibody fragments can be obtained using proteolytic enzymes or, preferably, produced by genetic recombination techniques.

The monoclonal antibody that specifically binds to mesothelin of the present invention can be prepared by using all or part of the mesothelin protein as an immunogen (or antigen). More specifically, as an immunogen, mesothelin, a fusion protein containing mesothelin protein, or a carrier containing mesothelin protein, if necessary, together with an adjuvant (e.g., Freund adjuvant), is injected once or more by subcutaneous, intramuscular, intravenous, intraperitoneal in mammals except for humans to achieve an immunization. The mammals other than humans are preferably mice, rats, hamsters, malmots, chickens, rabbits, cats, dogs, pigs, goats, sheep, donkeys, horses or cattle (including transgenic animals engineered to produce an antibody from other animals such as mice to produce human antibody), more preferably mouse, rat, hamster, malmot, chicken or rabbit. Antibody-producing cells can be obtained from the immune-sensitized mammal about 1 to 10 days after the final immunization by performing immunization 1 to 4 times every 1 to 21 days from the first immunization. The number of times and intervals for immunization can be appropriately changed depending on the characteristics of the immunogen to be used.

Preparation of a hybridoma secreting a monoclonal antibody can be carried out according to the method of Keira and Mirstein et al. (Nature, 1975, Vol. 256, p. 495-497) and a method similar thereto. Hybridomas can be produced by cell fusion of mammal-derived myeloma cells without autologous antibody-producing ability and antibody-producing cells contained in the group consisting of spleen, lymph node, bone marrow and tonsils, preferably spleen.

For cell fusion, for example, a fusion promoter including polyethylene glycol or Sendai virus or a method by electric pulse is used, for example, in a fusion medium containing a fusion promoter, antibody-producing cells and mammalian-derived cells capable of indefinite proliferation. Cells are suspended at a ratio of about 1:1 to 1:10, and in this state, cultured at about 30 to 40° C. for about 1 to 5 minutes. As the fusion medium, for example, MEM medium, RPM11640 medium, and Iscove's Modified Dulbecco's Medium may be used, and it is preferable to exclude sera such as bovine serum.

In the method of screening the hybridoma clones producing the monoclonal antibody, first, the fusion cells obtained as described above are transferred to a selection medium such as HAT medium, and cultured at about 30 to 40° C. for about 3 days to 3 weeks to kill cells other than hybridomas. Then, after culturing the hybridoma on a microtiter plate, etc., the part with increased reactivity between the immunogen used for the immune response of animals other than humans described above and the culture supernatant was subjected to RIA (radioactive substance-marked immuno antibody) or ELISA (Enzyme-Linked Immunosorbent Assay). The clone producing the monoclonal antibody found above shows specific binding ability to the immunogen.

The monoclonal antibody of the present invention can be obtained by culturing such a hybridoma in vitro or in vivo. For culturing, a conventional method for culturing cells derived from mammals is used, and for collecting monoclonal antibody from a culture or the like, a conventional method in this field for purifying an antibody in general is used. As each method, for example, salting out, dialysis, filtration, concentration, centrifugation, fractional precipitation, gel filtration chromatography, ion exchange chromatography, affinity chromatography, high-performance liquid chromatography, gel electrophoresis or isoelectric point electrophoresis, etc. can be applied, and these are applied in combination as needed. The purified monoclonal antibody is then concentrated and dried to be in a liquid or solid state depending on the use.

Furthermore, the monoclonal antibody of the present invention can be produced by synthesizing the linked gene in which DNA encoding the heavy chain and light chain variable regions are linked to DNA encoding the constant regions of the heavy and light chains (see, for example, Japanese Patent No. 2007-252372), respectively, by PCR or chemical synthesis; producing a transgene by transplanting it into a publicly available expression vector (pcDNA 3.1 (sold by Invitrogen), etc.) or the like that enables expression of the genes; producing antibodies by expressing them in a host such as CHO cells or E. coli; and purifying the antibodies from such cultures using a Protein A or G column or the like.

In a specific embodiment of the present invention, in order to prepare an antibody that specifically binds to mesothelin, hybridomas that produce an anti-mesothelin antibody are prepared and screened, and 7 kinds of antibodies (scFvs) that specifically bind to mesothelin were selected and designated as 3A8, 4G11, 5A9, 6G5, 7C3, 9E8, and 9E11, respectively.

(1) It was confirmed that 3A8 antibody includes a heavy chain variable region including a CDR1 region represented by an amino acid of SEQ ID NO: 1 (GYSFTGYT), a CDR2 region represented by amino acids of SEQ ID NO: 2 (INPYNGGT), and a CDR3 region represented by amino acids of SEQ ID NO: 3 (ARVGGSSWYFDV), and a light chain variable region including a CDR1 region represented by an amino acid of SEQ ID NO: 4 (QSLLYSSNQKNY), a CDR2 region with the amino acids WAS, and a CDR3 region represented by the amino acids of SEQ ID NO: 6 (QQGNTLPWT).

Specifically, 3A8 antibody contained a heavy chain variable region represented by the amino acid of SEQ ID NO: 7 and a light chain variable region represented by the amino acid of SEQ ID NO: 8, wherein the heavy chain variable region was encoded with the nucleotide sequence of SEQ ID NO: 9 and a light chain variable region was encoded with the nucleotide sequence of SEQ ID NO: 10.

(2) It was confirmed that 4G11 antibody comprises a CDR1 region represented by amino acids of SEQ ID NO: 11 (GYSFTGYY), a CDR2 region represented by amino acids of SEQ ID NO: 12 (ISCYNGAT) and a CDR3 region represented by amino acids of SEQ ID NO: 13 (ARWDRDWFAY), and a light chain variable region including a CDR1 region represented by an amino acid of SEQ ID NO: 14 (QDVGIA), a CDR2 region with amino acids: WAS, and a CDR3 region represented by amino acids of SEQ ID NO: 16 (QQYSSYPFT).

Specifically, 4G11 antibody contained a heavy chain variable region represented by the amino acid of SEQ ID NO: 17 and a light chain variable region represented by the amino acid of SEQ ID NO: 18, wherein the heavy chain variable region was encoded with the nucleotide sequence of SEQ ID NO: 19 and a light chain variable region was encoded with the nucleotide sequence of SEQ ID NO: 20.

(3) It was confirmed that 5A9 antibody had a heavy chain variable region including a CDR1 region represented by an amino acid of SEQ ID NO: 21 (GFSITSSSYC), a CDR2 region represented by amino acids of SEQ ID NO: 22 (ICYEGSI), and a CDR3 region represented by amino acids of SEQ ID NO: 23 (SRENRLLKDAMDY), and a light chain variable region including a CDR1 region represented by an amino acid of SEQ ID NO: 24 (QSLLSSRTRKNY), a CDR2 region with amino acids: WAS, and a CDR3 region represented by amino acids of SEQ ID NO: 26 (KQSYNLRT).

Specifically, the 5A9 antibody contained a heavy chain variable region represented by the amino acid of SEQ ID NO: 27 and a light chain variable region represented by the amino acid of SEQ ID NO: 28, wherein the heavy chain variable region was encoded with the nucleotide sequence of SEQ ID NO: 29 and a light chain variable region was encoded with the nucleotide sequence of SEQ ID NO: 30.

(4) It was confirmed that 6G5 antibody had a heavy chain variable region including a CDR1 region represented by an amino acid of SEQ ID NO: 31 (GYSFTGYT), a CDR2 region represented by amino acids of SEQ ID NO: 32 (INPYNGGT), and a CDR3 region represented by amino acids of SEQ ID NO: 33 (ARVGGSSWYFDV), and a light chain variable region including a CDR1 region represented by an amino acid of SEQ ID NO: 34 (QSLLYSSNQKNY), a CDR2 region with amino acids: WAS, and a CDR3 region represented by amino acids of SEQ ID NO: 36 (QQYYTYPTWT).

Specifically, 6G5 antibody contained a heavy chain variable region represented by the amino acid of SEQ ID NO: 37 and a light chain variable region represented by the amino acid of SEQ ID NO: 38, wherein the heavy chain variable region was encoded with the nucleotide sequence of SEQ ID NO: 39 and a light chain variable region was encoded with the nucleotide sequence of SEQ ID NO: 40.

(5) It was confirmed that 7C3 antibody had a heavy chain variable region including a CDR1 region represented by an amino acid of SEQ ID NO: 41 (GYTFSAYW), a CDR2 region represented by amino acids of SEQ ID NO: 42 (ILPGSGST), and a CDR3 region represented by amino acids of SEQ ID NO: 43 (ARGDYYAMDY), and a light chain variable region including a CDR1 region represented by an amino acid of SEQ ID NO: 44 (QSLLYSNGKTY), a CDR2 region with amino acids: LVS, and a CDR3 region represented by amino acids of SEQ ID NO: 46 (VQGTHFPFT).

Specifically, 7C3 antibody contained a heavy chain variable region represented by the amino acid of SEQ ID NO: 47 and a light chain variable region represented by the amino acid of SEQ ID NO: 48, wherein the heavy chain variable region was encoded with the nucleotide sequence of SEQ ID NO: 49 and a light chain variable region was encoded with the nucleotide sequence of SEQ ID NO: 50.

(6) It was confirmed that 9E8 antibody had a heavy chain variable region including a CDR1 region represented by an amino acid of SEQ ID NO: 51 (GYSFTGYT), a CDR2 region represented by amino acids of SEQ ID NO: 52 (INPYNGGT), and a CDR3 region represented by amino acids of SEQ ID NO: 53 (ARVGGSSWYFDV), and a light chain variable region including a CDR1 region represented by an amino acid of SEQ ID NO: 54 (QSLLYSSNQKNY), a CDR2 region with amino acids: WAS, and a CDR3 region represented by amino acids of SEQ ID NO: 56 (QQYYSYPTWT).

Specifically, 9E8 antibody contained a heavy chain variable region represented by the amino acid of SEQ ID NO: 57 and a light chain variable region represented by the amino acid of SEQ ID NO: 58, wherein the heavy chain variable region was encoded with the nucleotide sequence of SEQ ID NO: 59 and a light chain variable region was encoded with the nucleotide sequence of SEQ ID NO: 60.

(7) It was confirmed that 9E11 antibody had a heavy chain variable region including a CDR1 region represented by an amino acid of SEQ ID NO: 61 (GYSITSDYA), a CDR2 region represented by amino acids of SEQ ID NO: 62 (ISYSGST), and a CDR3 region represented by amino acids of SEQ ID NO: 63 (ARGAAGFAY), and a light chain variable region including a CDR1 region represented by an amino acid of SEQ ID NO: 64 (QTIGTW), a CDR2 region with amino acids: AAT, and a CDR3 region represented by amino acids of SEQ ID NO: 66 (QQLYSTPWT).

Specifically, 9E11 antibody contained a heavy chain variable region represented by the amino acid of SEQ ID NO: 67 and a light chain variable region represented by the amino acid of SEQ ID NO: 68, wherein the heavy chain variable region was encoded with the nucleotide sequence of SEQ ID NO: 69 and a light chain variable region was encoded with the nucleotide sequence of SEQ ID NO: 70.

The antibody specific for mesothelin of the present invention is preferably scFv (single chain variable fragment), and can be produced through genetic recombination technology so that the heavy chain variable region and a light chain variable region can be linked with a linker. The linker may preferably be represented by the amino acid sequence of SEQ ID NO: 71 or the nucleotide sequence of SEQ ID NO: 72, but is not limited thereto.

When linked by a light chain variable region-linker-a heavy chain variable region, 3A8 antibody has the amino acid sequence of SEQ ID NO: 73 or the nucleotide sequence of SEQ ID NO: 74, and 4G11 antibody has the amino acid sequence of SEQ ID NO: 75 or the nucleotide sequence of SEQ ID NO: 76, 5A9 antibody has the amino acid sequence of SEQ ID NO: 67 or the nucleotide sequence of SEQ ID NO: 78, and 6G5 antibody has the amino acid sequence of SEQ ID NO: 79 or the nucleotide sequence of SEQ ID NO: 80, 7C3 antibody has the amino acid sequence of SEQ ID NO: 81 or the nucleotide sequence of SEQ ID NO: 82, 9E8 antibody has the amino acid sequence of SEQ ID NO: 83 or the nucleotide sequence of SEQ ID NO: 84, and 9E11 antibody has the amino acid sequence of SEQ ID NO: 85 or the nucleotide sequence of SEQ ID NO: 86.

In another aspect, the present invention relates to a polynucleotide encoding the antibody that specifically binds to mesothelin.

As used herein, the term “polynucleotide” generally refers to a nucleic acid molecule, deoxyribonucleotide or ribonucleotide, or an analog thereof, separated by any length. In some embodiments, a polynucleotide of the present invention can be prepared by (1) in-vitro amplification, such as polymerase chain reaction (PCR) amplification; (2) cloning and recombination; (3) purification such as digestion and gel electrophoretic separation; (4) synthesis such as chemical synthesis, and preferably, the isolated polynucleotide is prepared by recombinant DNA technology. In the present invention, the nucleic acid for encoding the antibody or antigen-binding fragment thereof can be prepared by various methods known in the art, including, but not limited to, restriction fragment operation of synthetic oligonucleotides or application of SOE PCR.

In another aspect, the present invention relates to a vector comprising the polynucleotide encoding the antibody that specifically binds to mesothelin, and a recombinant cell transformed with the vector.

In the present invention, the term “vector (expression vector)” refers to a gene preparation including essential regulatory elements such as a promoter so that a target gene can be expressed in an appropriate host cell. A vector may be selected from one or more of a plasmid, a retroviral vector, and a lentiviral vector. Upon transformation into an appropriate host, a vector can replicate and function independently of the host genome, or in some cases can be integrated into the genome itself.

In addition, a vector may contain expression control elements that allow the coding region to be accurately expressed in a suitable host. Such regulatory elements are well known to those skilled in the art and include, for example, promoters, ribosome-binding sites, enhancers and other regulatory elements for regulating gene transcription or mRNA translation. The specific structure of the expression control sequence may vary depending on the function of the species or cell type, but generally contains 5′ non-translated sequence, and a 5′ or 3′ non-translated sequence participating in transcription initiation and translation initiation, respectively, such as TATA box, capped sequence, CAAT sequence, etc. For example, a 5′ non-transcriptional expression control sequence can include a promoter region that can include a promoter sequence for transcription and control of a functionally linked nucleic acid.

As used herein, the term “promoter” means a minimal sequence sufficient to direct transcription. In addition, promoter constructs sufficient to allow expression of a regulatable promoter-dependent gene induced by cell type-specific or external signals or agents may be included, and these constructs may be located in the 5′ or 3′ portion of the gene. Both conservative and inducible promoters are included. Promoter sequences may be derived from prokaryotes, eukaryotes or viruses.

In the present invention, the term “transformant” refers to a cell transformed by introducing a vector having a polynucleotide encoding one or more target proteins into a host cell, and a method for introducing the expression a vector into the host cell to form a transformant are such as a calcium phosphate method or a calcium chloride/rubidium chloride method, an electroporation method, an electroinjection method, a chemical treatment method such as PEG, a method using a gene gun, and the like (Sambrook, J., et al., Molecular Cloning, A Laboratory Manual(2^{nd} ed.), Cold Spring Harbor Laboratory, 1. 74, 1989).

When the transformant expressing the vector is cultured in a nutrient medium, an antibody protein can be produced and isolated in large quantities. Medium and culture conditions can be appropriately selected and used depending on the host cell. During culture, conditions such as temperature, medium pH, and culture time should be appropriately adjusted to be suitable for cell growth and mass production of proteins.

The vector according to the present invention can be transformed into a host cell, preferably a mammalian cell, for the production of the antibody. Suitable host cells capable of expressing fully glycosylated proteins include COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Agl4, 293 cells, HeLa cells, etc., and these cells are readily available from, for example, ATCC (American Type Culture Collection, USA).

### Chimeric Antigen Receptor Targeting Mesothelin

From another point of view, the present invention also relates to a chimeric antigen receptor (CAR) targeting mesothelin comprising: a mesothelin-binding domain; a transmembrane domain; a costimulatory domain; and an intracellular signal transduction domain,
wherein the mesothelin-binding domain is an antibody specifically binding to mesothelin or a fragment thereof, comprising:
(1) a heavy chain variable region including a CDR1 region represented by an amino acid of SEQ ID NO: 1, a CDR2 region represented by an amino acid of SEQ ID NO: 2 and a CDR3 region represented by an amino acid of SEQ ID NO: 3, and a light chain variable region including a CDR1 region represented by an amino acid of SEQ ID NO: 4, a CDR2 region with an amino acid sequence: WAS and a CDR3 region represented by an amino acid of SEQ ID NO: 6;
(2) a heavy chain variable region including a CDR1 region represented by an amino acid of SEQ ID NO: 11, a CDR2 region represented by an amino acid of SEQ ID NO: 12 and a CDR3 region represented by an amino acid of SEQ ID NO: 13, and a light chain variable region including a CDR1 region represented by an amino acid of SEQ ID NO: 14, a CDR2 region with an amino acid sequence: WAS and a CDR3 region represented by an amino acid of SEQ ID NO: 16;
(3) a heavy chain variable region including a CDR1 region represented by an amino acid of SEQ ID NO: 21, a CDR2 region represented by an amino acid of SEQ ID NO: 22 and a CDR3 region represented by an amino acid of SEQ ID NO: 23, and a light chain variable region including a CDR1 region represented by an amino acid of SEQ ID NO: 24, a CDR2 region with an amino acid sequence: WAS and a CDR3 region represented by an amino acid of SEQ ID NO: 26;
(4) a heavy chain variable region including a CDR1 region represented by an amino acid of SEQ ID NO: 31, a CDR2 region represented by an amino acid of SEQ ID NO: 32 and a CDR3 region represented by an amino acid of SEQ ID NO: 33, and a light chain variable region including a CDR1 region represented by an amino acid of SEQ ID NO: 34, a CDR2 region with an amino acid of WAS and a CDR3 region represented by an amino acid of SEQ ID NO: 36;
(5) a heavy chain variable region including a CDR1 region represented by an amino acid of SEQ ID NO: 41, a CDR2 region represented by an amino acid of SEQ ID NO: 42 and a CDR3 region represented by an amino acid of SEQ ID NO: 43, and a light chain variable region including a CDR1 region represented by an amino acid of SEQ ID NO: 44, a CDR2 region with an amino acid sequence of LVS and a CDR3 region represented by an amino acid of SEQ ID NO: 46;
(6) a heavy chain variable region including a CDR1 region represented by an amino acid of SEQ ID NO: 51, a CDR2 region represented by an amino acid of SEQ ID NO: 52 and a CDR3 region represented by an amino acid of SEQ ID NO: 53, and a light chain variable region including a CDR1 region represented by an amino acid of SEQ ID NO: 54, a CDR2 region with an amino acid sequence of WAS and a CDR3 region represented by an amino acid of SEQ ID NO: 56; or
(7) a heavy chain variable region including a CDR1 region represented by an amino acid of SEQ ID NO: 61, a CDR2 region represented by an amino acid of SEQ ID NO: 62 and a CDR3 region represented by an amino acid of SEQ ID NO: 63, and a light chain variable region including a CDR1 region represented by an amino acid of SEQ ID NO: 64, a CDR2 region with an amino acid sequence of AAT and a CDR3 region represented by an amino acid of SEQ ID NO: 66.

As used herein, the term “chimeric antigen receptor (CAR)” generally refers to a fusion protein containing an extracellular domain having the ability to bind an antigen and one or more intracellular domains. A CAR is a core part of a chimeric antigen receptor T cell (CAR-T) and may contain an antigen (e.g., mesothelin) binding domain, a transmembrane domain, a co-stimulatory domain, and an intracellular signal transduction domain. A CAR can be combined with a T cell receptor-activating intracellular domain based on the antigen specificity of the antibody. Genetically modified CAR-expressing T cells can specifically identify and eliminate target antigen-expressing malignant cells.

In the present invention, the term “mesothelin-binding domain” generally refers to a domain capable of specifically binding to a mesothelin protein. For example, the mesothelin-binding domain may comprise an anti-mesothelin antibody or fragment thereof that can specifically bind to a mesothelin polypeptide or fragment thereof that is overexpressed in a cancer or tumor cell.

In the present invention, the term “binding domain” can be used interchangeably refers to “extracellular domain”, “extracellular binding domain”, “antigen-specific binding domain” and “extracellular antigen-specific binding domain” and refers to a CAR domain or fragment that has the ability to specifically bind to a target antigen (e.g., mesothelin).

In the present invention, anti-mesothelin antibody or a fragment thereof is the aforementioned anti-mesothelin antibody, a monoclonal antibody, preferably a single chain variable fragment (scFv). Specifically, it can be prepared using the mesothelin-specific 3A8, 4G11, 5A9, 6G5, 7C3, 9E8, and 9E11 antibodies of the present invention, preferably 3A8, 4G11, 5A9, 6G5, and 7C3 antibodies.

In the present invention, a signal peptide may be further included at the N-terminus of the mesothelin-binding domain, and the “signal peptide” generally refers to a peptide chain for guiding protein transduction. The signal peptide may be a short peptide having a length of 5 to 30 amino acids, preferably represented by the amino acid sequence of SEQ ID NO: 94.

In the present invention, it may further include a hinge region located between the C terminus of the mesothelin-binding domain and the N terminus of a transmembrane domain, wherein the hinge region is derived from CD8a, and preferably, it can be represented by the amino acid sequence of SEQ ID NO: 95. The “hinge region” generally refers to the linking region between an antigen-binding region and an immune cell Fc receptor (FcR)-binding region.

In the present invention, “a transmembrane domain” refers to a domain of a CAR that generally passes through a cell membrane and is connected to an intracellular signal transduction domain to play a role in signal transduction. The transmembrane domain may be derived from a protein selected from the group consisting of CD8α, CD4, CD28, CD137, CD80, CD86, CD152 and PD1, and preferably may be represented by the amino acid sequence of SEQ ID NO: 96.

In the present invention, “costimulatory domain” generally refers to an intracellular domain capable of providing immune-stimulatory molecules, which are cell surface molecules necessary for an effective response of lymphocytes to antigens. The costimulatory domain described above may comprise a costimulatory domain of CD28, and may comprise a costimulatory domain of the TNF receptor family, such as the costimulatory domain of OX40 and 4-1BB, preferably it may be 4-1BB represented by the amino acid sequence of SEQ ID NO: 97.

In the present invention, “intracellular signal transduction domain” generally refers to a domain located inside a cell and capable of transmitting a signal. In the present invention, the intracellular signal transduction domain is an intracellular signal transduction domain of the chimeric antigen receptor. For example, the intracellular signal transduction domain may be selected from CD3ζ intracellular domain, CD28 intracellular domain, CD28 intracellular domain, 4-1BB intracellular domain and OX40 intracellular domain, and preferably it may be CD3ζ represented by the amino acid sequence of SEQ ID NO: 98.

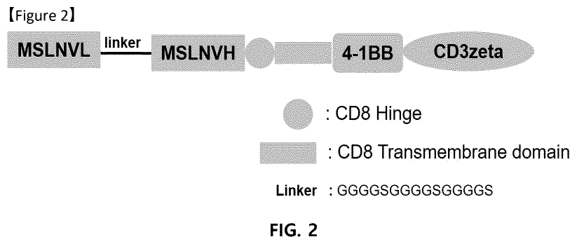

The chimeric antigen receptor targeting mesothelin of the present invention (MSLN-CAR) can be preferably prepared as shown in the schematic diagram shown in FIG. 2.

### Polynucleotide Coding Chimeric Antigen Receptor and Vector Expressing Chimeric Antigen Receptor

In another aspect, the present invention relates to a polynucleotide encoding the chimeric antigen receptor targeting mesothelin (MSLN-CAR).

In the present invention, the polynucleotide encoding a chimeric antigen receptor targeting mesothelin (MSLN-CAR) is a polynucleotide encoding a mesothelin-binding domain; a polynucleotide encoding a transmembrane domain; a polynucleotide encoding a costimulatory domain; and a polynucleotide encoding an intracellular signal transduction domain.

A polynucleotide encoding the mesothelin-binding domain may be a polynucleotide encoding the antibody specific for mesothelin of the present invention, 3A8, 4G11, 5A9, 6G5, 7C3, 9E8 and/or 9E11, preferably 3A8, 4G11, 5A9, 6G5, and/or 7C3, and a light chain variable region and a heavy chain variable region may be in the form of scFv linked by a linker, and the specific nucleotide sequence may be the same as described above.

Preferably, a polynucleotide encoding a chimeric antigen receptor (CAR) of the present invention may has:
a signal peptide represented by the nucleotide sequence of SEQ ID NO: 88;
3A8 antibody represented by the nucleotide sequence of SEQ ID NO: 74, 4G11 antibody represented by the nucleotide sequence of SEQ ID NO: 76, 6G5 antibody represented by the nucleotide sequence of SEQ ID NO: 80, or 7C3 antibody represented by the nucleotide sequence of SEQ ID NO: 82;
a transmembrane domain represented by the nucleotide sequence of 90;
4-1BB (a costimulatory domain) represented by the nucleotide sequence of SEQ ID NO: 91; and
an intracellular signal transduction domain (CD3ζ represented by the nucleotide sequence of SEQ ID NO: 92.

In addition, a polynucleotide encoding a hinge region may be additionally included between a polynucleotide encoding the mesothelin-binding domain and a transmembrane domain, and preferably It may be a CD8 hinge region represented by the nucleotide sequence of SEQ ID NO: 89.

In another aspect, the present invention relates to a vector comprising a polynucleotide encoding the chimeric antigen receptor targeting mesothelin (MSLN-CAR).

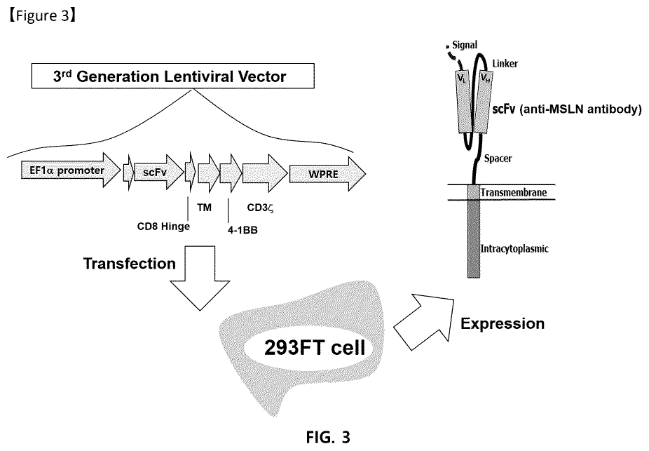

In a specific embodiment of the present invention, the vector is a recombinant virus a vector, preferably a lentivirus vector, and comprises an operably linked EF1α promoter; a polynucleotide encoding a signal peptide; a polynucleotide encoding a mesothelin-binding domain; a polynucleotide encoding a transmembrane domain; and a polynucleotide encoding an intracellular signal transduction domain, and may further include a woodchuck hepatitis virus post-transcriptional regulatory element (WPRE) to increase protein expression (refer to FIG. 3).

The EF1α promoter may be represented by the sequence of SEQ ID NO: 87, and may include a sequence that is 90% or more, 93% or more, 95% or more, 96% or more, 97% or more, 98% or more, or 99% or more identical to the sequence of SEQ ID NO: 87, as desired.

In addition, the promoter is operably linked to induce expression of an anti-mesothelin antibody (scFv), which is a mesothelin-binding domain.

Biological methods for introducing polynucleotides into host cells include the use of DNA and RNA vectors. Viral vectors, and in particular retroviral vectors, have become the most widely used methods for inserting genes into mammalian, e.g., human cells. Other virus vector may be derived from lentiviruses, poxviruses, herpes simplex viruses, adenoviruses and adeno-associated viruses, and the like.

Chemical means for introducing polynucleotides into host cells include colloidal dispersion systems such as macromolecular complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods are available for state-of-the-art targeted delivery of nucleic acids, e.g., delivery of polynucleotides using targeted nanoparticles or other suitable sub-micrometer-sized delivery systems.

When a non-viral delivery system is used, an exemplary delivery vehicle is a liposome. The use of lipid preparations is contemplated for the introduction of nucleic acids into host cells (in vitro, ex vivo or in vivo). In another aspect, the nucleic acid may be associated with a lipid. Nucleic acids associated with lipids may be encapsulated within the aqueous interior of the liposome, interspersed within the lipid bilayer of the liposome, attached to the liposome via a linking molecule associated with both the liposome and oligonucleotide, captured within the liposome, complexed with the liposome, dispersed in a lipid-containing solution, mixed with a lipid or combined with a lipid, contained as a suspension in a lipid, contained or complexed with micelles, or otherwise associated with a lipid. Lipid, lipid/DNA or lipid/expression a vector association composition is not limited to any particular structure in solution.

### Immune Effector Cell Expressing Chimeric Antigen Receptor (CAR)

In another aspect, the present invention relates to an immune effector cell expressing the chimeric antigen receptor targeting mesothelin (MSLN-CAR) includes a vector comprising a polynucleotide encoding a chimeric antigen receptor, or a polynucleotide encoding a chimeric antigen receptor.

In the present invention, the immune effector cell may be a mammalian-derived cell, preferably T cells, B cells, natural killer (NK) cells, dendritic cells, myeloid cells, monocytes, or macrophages, more preferably T cells.

In the present invention, an immune effector cell expressing MSLN-CAR can be prepared by introducing the MSLN-CAR expression vector of the present invention into an immune effector cell, for example, a T cell or NK cell.

Specifically, the MSLN-CAR expression vector can be introduced into cells by methods known in the art, such as electroporation, lipofectamine (lipofectamine 2000, Invitrogen), and the like. For example, an immune effector cell can be transfected by a lentiviral vector to integrate the viral genome carrying the CAR molecule into the host genome to ensure long-term and stable expression of the target gene. For another example, a transposon can be used to introduce a CAR transport plasmid and a transferase transport plasmid into a target cell. For another example, a CAR molecule can be added to the genome by a gene editing method (e.g., CRISPRCas9).

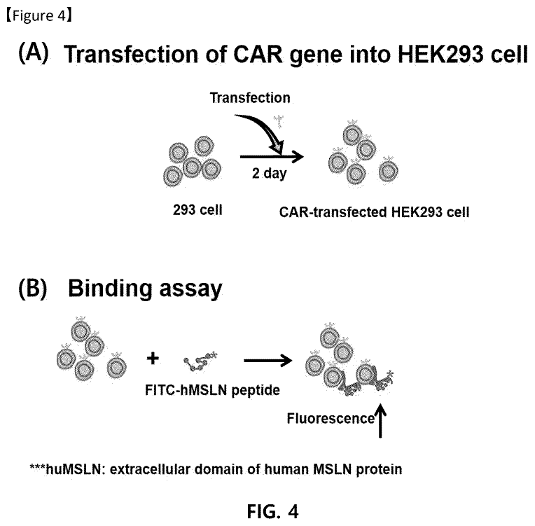

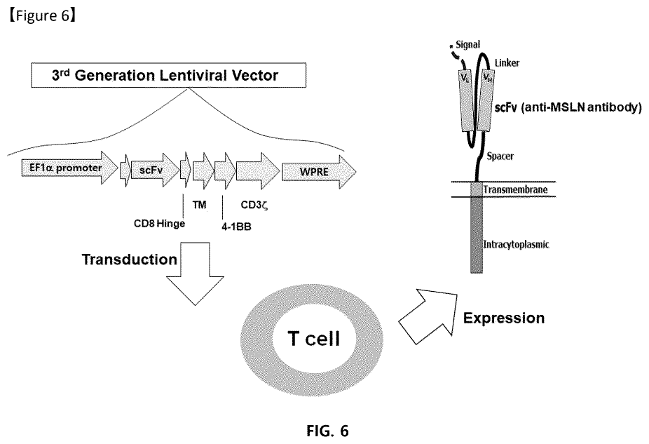

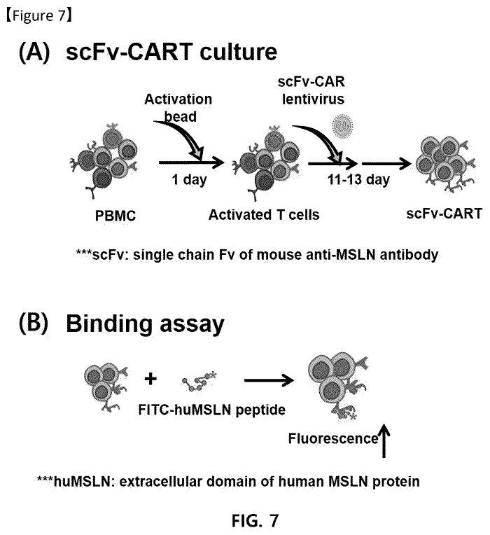

In a specific embodiment of the present invention, a lentiviral vector containing a polynucleotide encoding MSLN-CAR was prepared as shown in FIGS. 3 and 4, and the prepared vector was transduced into T cells to produce MSLN-CAR-T cells (FIGS. 6 and 7). The prepared MSLN-CAR-T cells express a chimeric antigen receptor targeting the mesothelin of the present invention.

An immune effector cell for the production of immune effector cell expressing a chimeric antigen receptor (CAR) can be obtained from a subject, wherein the “subject” includes a living organism (e.g., a mammal from which an immune response can be elicited). Examples of subjects include humans, dogs, cats, mice, rats, and transgenic species thereof. T cells can be obtained from numerous sources, including peripheral blood mononuclear cells, bone marrow, lymph node tissue, umbilical cord blood, thymus tissue, tissue from the site of infection, ascites, pleural effusion, splenic tissue, and tumors.

Such T cells can be obtained from blood units collected from a subject using any of a number of techniques known to those of ordinary skill in the art, for example, Ficoll™ isolation. Cells from blood are obtained by apheresis, and apheresis products typically contain T cells, monocytes, granulocytes, lymphocytes including B cells, other nucleated leukocytes, red blood cells, and platelets.

Cells collected by apheresis can be washed to remove the plasma fraction and place the cells in an appropriate buffer or medium forsubsequent processing steps. Tcells are isolated from peripheral blood lymphocytes by lysing red blood cells and depleting monocytes, for example by centrifugation through a PERCOLL™ gradient or by countercurrent centrifugation.

In a specific embodiment of the present invention, as shown in FIGS. 6 and 7, after isolating activated T cells from peripheral blood mononuclear cells (PBMCs), MSLN-CAR lentivirus was transduced into T cells to prepare MSLN-CAR-T cells, and specifically, MSLN-CAR-T cells were prepared using 3A8, 4G11, 6G5, and 7C3 antibodies, respectively.

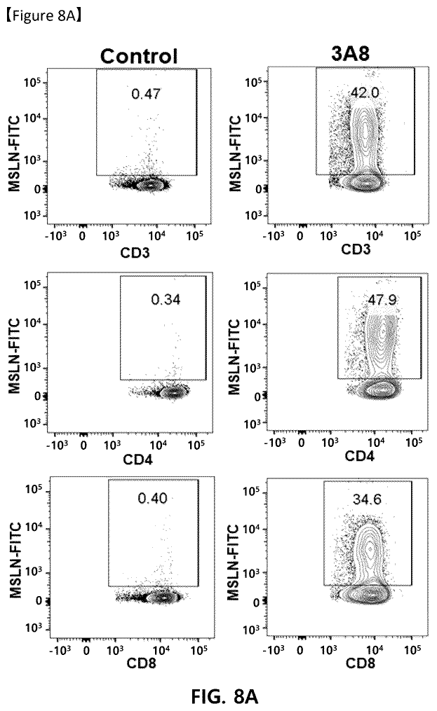

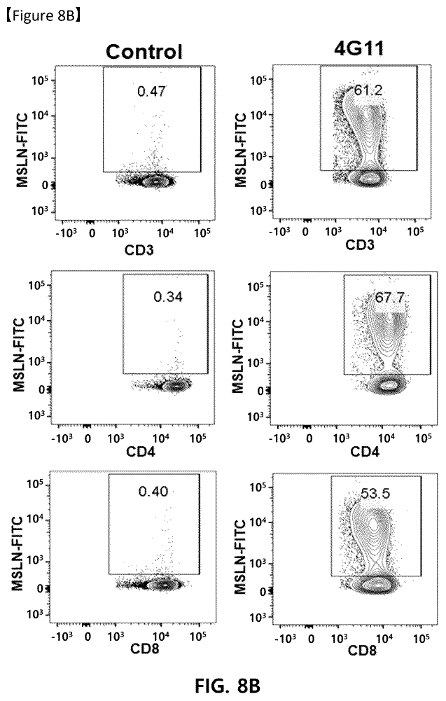

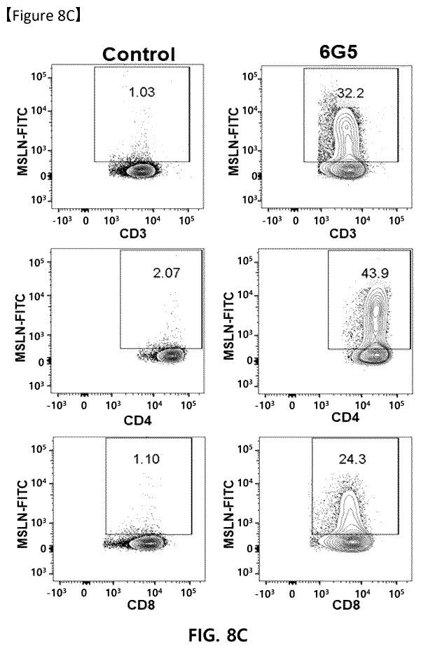

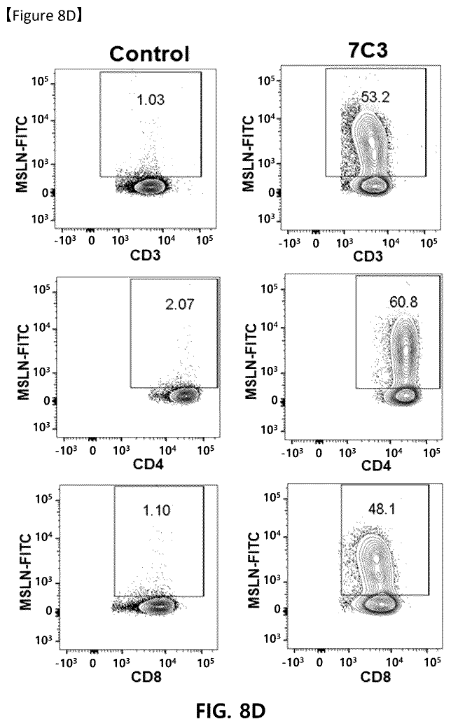

In order to confirm the activity of the prepared MSLN-CAR-T cells, the binding ability to mesothelin peptides for CD3-, CD4-, or CD8-activated MSLN-CAR-T cells was confirmed. As shown in FIGS. 8A to 8D, it was confirmed that the MSLN-CAR-T cells prepared in the present invention bound to the mesothelin peptide.

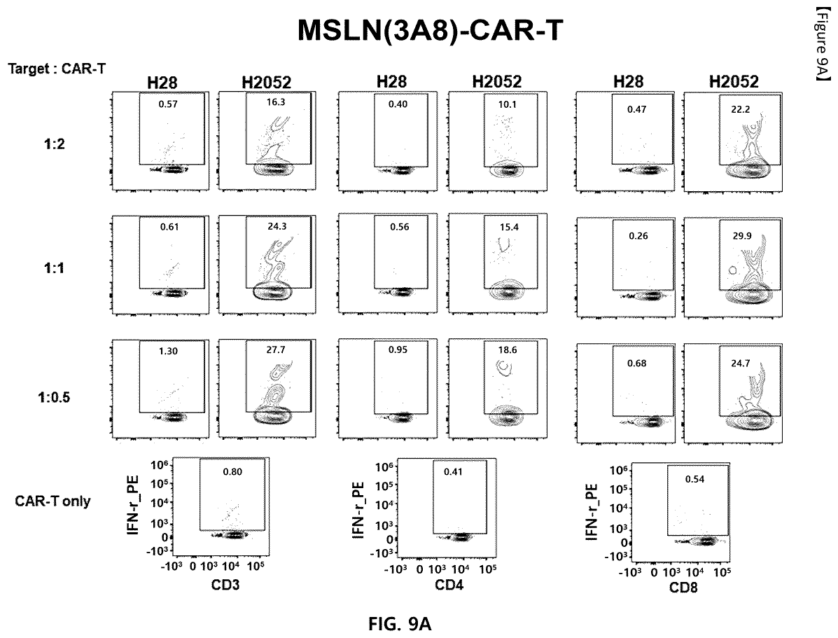

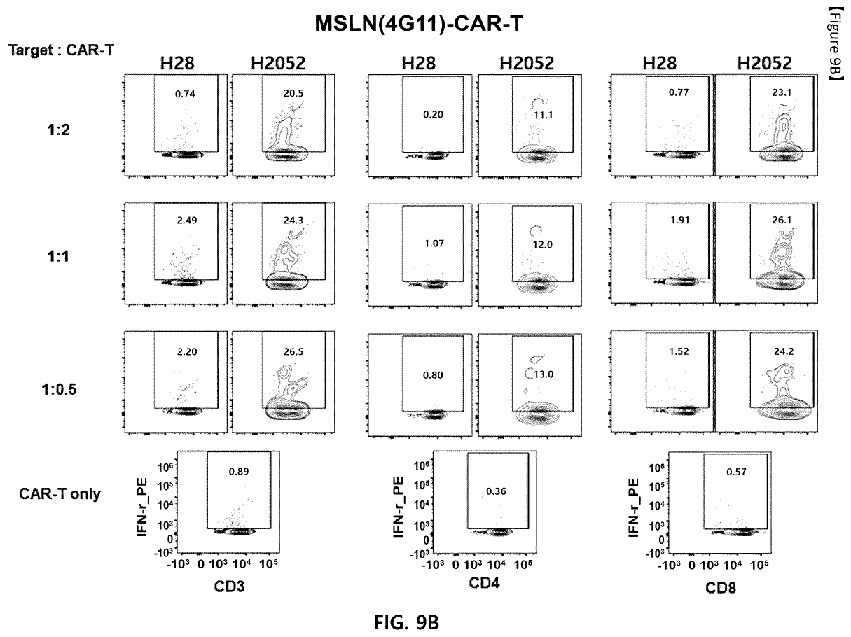

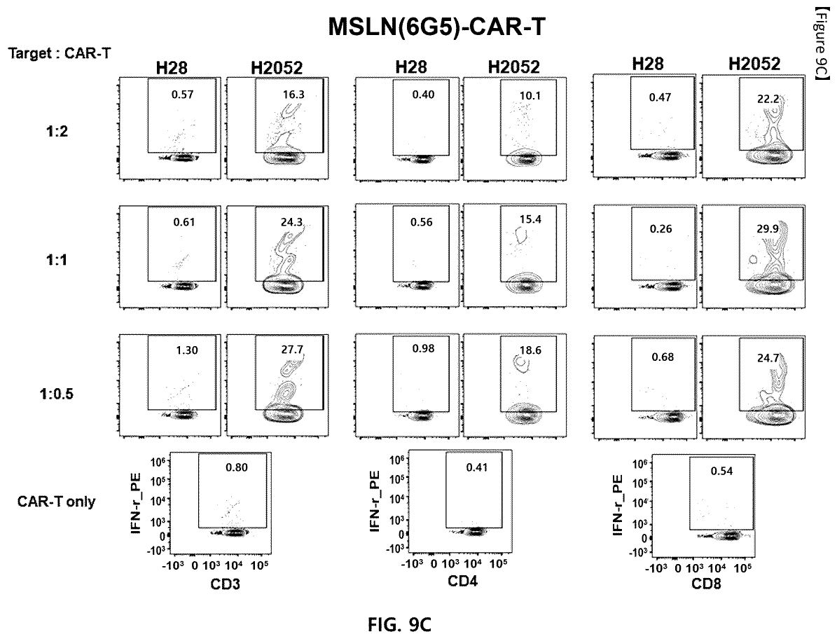

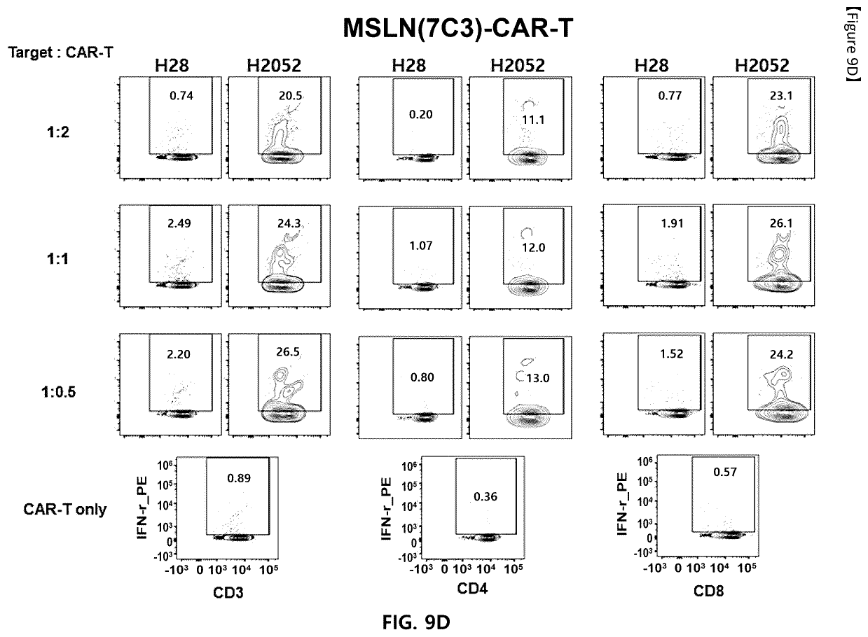

In another specific embodiment of the present invention, in order to confirm activation of MSLN-CAR-T cells, the level of IFNγ expression by MSLN-CAR-T cells in the presence of target cells was confirmed. As a result, as shown in FIGS. 9A to 9D, it was confirmed that T cells were not activated in H28 cells not expressing mesothelin, whereas T cells were activated in the presence of H2052 cells overexpressing mesothelin, resulting in increased IFNγ expression

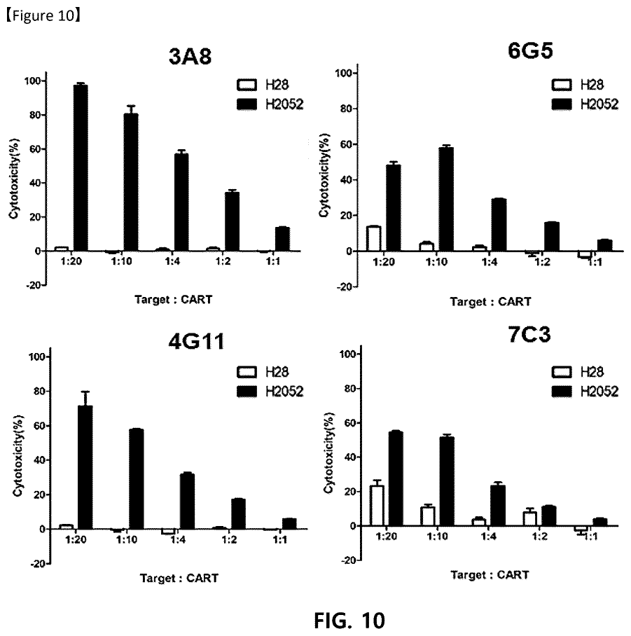

In another specific embodiment of the present invention, as a result of confirming the killing effect of target cells by MSLN-CAR-T cells, as shown in FIG. 10, the MSLN-CAR-T cells exhibited a killing effect specific to H2052 cells overexpressing mesothelin.

That is, the antibodies selected in the present invention specifically recognized mesothelin-overexpressing cancer or tumor cells, and thus could effectively induce cytotoxicity or killing by immune cells/macrophages by inhibiting immune evasion of mesothelin-overexpressing cancer or tumor cells. Furthermore, since the MSLN-CAR-T cells produced by the present invention were found to have a specific killing effect on mesothelin-overexpressing cells, the anti-mesothelin antibody of the present invention and the CAR-T cells using the same may be useful as compositions for the prevention or treatment of diseases associated with mesothelin overexpression, in particular, cancer or tumors.

### Composition for Preventing or Treating Diseases Mediated by Mesothelin Expression

In another aspect, the present invention includes a pharmaceutical composition for use in preventing or treating a cancer or tumor expressing mesothelin, comprising: the antibody specifically binding to mesothelin or the fragment thereof; or the immune effector cell expressing a chimeric antigen receptor targeting mesothelin.

In the present invention, the cancer or tumor may be selected from the group consisting of squamous cell carcinoma, small cell lung cancer, non-small cell lung cancer, adenocarcinoma of the lung, squamous cell carcinoma of the lung, mesothelioma, peritoneal cancer, hepatocellular carcinoma, gastrointestinal cancer, pancreatic cancer, glioma, cervical cancer, ovarian cancer, liver cancer, bladder cancer, hepatocellular carcinoma, breast cancer, colon cancer, colorectal cancer, endometrial or uterine carcinoma, salivary gland carcinoma, kidney cancer, liver cancer, prostate cancer, vulvar cancer, thyroid cancer, liver carcinoma, leukemia and other lymphoproliferative disorders, and various types of head and neck cancer.

In the present invention, the composition may include a therapeutic agent for a disease mediated by cells expressing mesothelin, wherein the therapeutic agent may be covalently bound to the heavy and/or light chain of antibody that specifically binds to mesothelin, it can be administered in combination with antibody or MSLN-CAR-T cell specific for mesothelin of the present invention

In addition, the therapeutic agent may be an anticancer agent. Anticancer agents reduce the proliferation of cancer cells and include non-peptidyl (i.e., non-protein) compounds, including cytotoxic agents and cytostatic agents. Non-limiting examples of anticancer agents include alkylating agents, nitrosourea, antimetabolites, antitumor antibiotics, plant (vinca) alkaloids, and steroid hormones. Peptide compounds may also be used.

In the pharmaceutical composition, the antibody that specifically binds to mesothelin or an immune effector cell expressing a chimeric antigen receptor targeting mesothelin may be the only active ingredient in the composition for treatment or diagnosis, or, can be used with other active ingredients for example, an anti-T cell, other antibody components such as anti-IFNγ or anti-LPS antibody, or non-antibody components such as xanthine.

The pharmaceutical composition preferably contains a therapeutically effective amount of antibody of the present invention. As used herein, the term “therapeutically effective amount” refers to an amount of a therapeutic agent required to treat, ameliorate, or prevent a target disease or condition, or the amount of a therapeutic agent required to exhibit a detectable therapeutic or prophylactic effect. For any antibody, a therapeutically effective dose can be initially determined by cell culture assays or animal models, usually rodents, rabbits, dogs, pigs, or primates. Animal models can also be used to determine appropriate concentration ranges and routes of administration. Such information can be used to determine useful dosages and routes for dosing in humans.

The precise effective amount for a human patient can vary depending on the severity of the disease state, the patient's general health, the patient's age, weight and sex, diet, administration time, administration frequency, drug composition, response sensitivity, and tolerance/response to treatment. The amount can be determined by routine experimentation and is within the scope of the clinician's judgment. In general, an effective dosage is 0.01-50 mg/kg, preferably 0.1-20 mg/kg, more preferably about 15 mg/kg.

The compositions may be administered to the patient individually or in combination with other preparations, agents, or hormones.

The dosage at which the antibody of the present invention is administered depends on the nature of the condition to be treated, the grade of malignant lymphoma or leukemia, and whether the antibody is used to prevent disease or to treat an existing condition.

The frequency of administration depends on the half-life of the antibody molecule and the duration of the drug's effect. If an antibody molecule has a short half-life (e.g., 2 to 10 hours), it may be necessary to provide one or more doses per day. Alternatively, if an antibody molecule has a long half-life (e.g., 2 to 15 days), it may be necessary to provide a dose once a day, once a week, or once every 1 or 2 months.

In addition, the pharmaceutical composition may contain a pharmaceutically acceptable carrier for administration of an antibody. The carrier itself must not cause the production of an antibody that is harmful to the subject receiving the composition, and must be non-toxic. Suitable carriers may be slowly metabolized macromolecules, such as proteins, polypeptides, liposomes, polysaccharides, polylactic acid, polyglycolic acid, amino acid polymers, amino acid copolymers and inactive viral particles.

A pharmaceutically acceptable salt may be used, and it contains for example, mineral acid salts such as hydrochloride, hydrobromide, phosphate and sulfate, or salts of organic acids such as acetic acid, propionic acid, malonic acid and benzoic acid.

A pharmaceutically acceptable carrier in therapeutic compositions may additionally include liquids such as water, saline, glycerol and ethanol. Additionally, auxiliary substances such as wetting agents, emulsifying agents or pH buffering agents may be present in such compositions. The carrier may be formulated as tablets, pills, sugar-coated tablets, capsules, liquids, gels, syrups, slurries and suspensions for ingestion of the pharmaceutical composition by a patient.

Preferred forms for administration may include those suitable for parenteral administration, for example by injection or infusion (e.g., bolus injection or continuous infusion). When the product is intended for infusion or injection, it may take the form of suspensions, solutions or emulsions in oil or water-soluble excipients, which may contain prescription agents such as suspending, preservative, stabilizing and/or dispersing agents. Alternatively, an antibody molecule may be in anhydrous form and reconstituted with an appropriate sterile solution prior to use.

Once formulated, the compositions of the present invention can be administered directly to a patient. The patients to be treated may be animals. However, the composition is preferably adapted for administration to human patients.

The pharmaceutical composition of the invention can be administered by any route, including, but not limited to, oral, intravenous, intramuscular, intra-arterial, intramedullary, intrathecal, intraspinal, intraventricular, transdermal, transcutaneous (see, e.g., WO 98/20734), subcutaneous, intraperitoneal, intranasal, enteric, topical, sublingual, vaginal, or rectal routes. Hyposprays may be used to administer the pharmaceutical compositions of the present invention. Typically, the therapeutic composition may be formulated as an injectable material as a liquid solution or suspension. Additionally, a solid form suitable for a liquid excipient solution or suspension may be prepared prior to injection.

Direct delivery of the composition may generally be achieved by injection, subcutaneous injection, intraperitoneal injection, intravenous injection, intramuscular injection, or may be delivered to the interstitial space of a tissue. In addition, the composition may be administered to the wound site. Dosage treatment may be a single dose schedule or a multiple dose schedule.

The active ingredient in the composition may be an antibody molecule. As such, it may be susceptible to degradation in the gastrointestinal tract. Thus, if the composition is administered by a route using the gastrointestinal tract, the composition may need to contain an agent that protects the antibody from degradation but releases the antibody once absorbed from the gastrointestinal tract.

A complete discussion of pharmaceutically acceptable carriers is available in Remington's Pharmaceutical Sciences (Mack Publishing Company, NJ, 1991).

### Diagnosis or Monitoring of Diseases Mediated by Cells Expressing Mesothelin

In another aspect, the present invention relates to a composition for diagnosing or monitoring a disease mediated by cells expressing mesothelin, comprising the antibody that specifically binds to mesothelin.

The mesothelin-overexpressing cancer or tumor may be a cancer or tumor in which mesothelin is overexpressed relative to normal controls or normal cells, specifically squamous cell carcinoma, small cell lung cancer, non-small cell lung cancer, adenocarcinoma of the lung, squamous cell carcinoma of the lung, mesothelial cancer, peritoneal cancer, hepatocellular carcinoma, gastrointestinal cancer, pancreatic cancer, glioma, cervical cancer, ovarian cancer, liver cancer, bladder cancer, hepatocellular carcinoma, breast cancer, colon cancer, colorectal cancer, endometrial or uterine carcinoma, salivary gland carcinoma, kidney cancer, liver cancer, prostate cancer, vulvar cancer, thyroid cancer, liver carcinoma, leukemia and other lymphoproliferative disorders, and various types of head and neck cancer.

The antibody that specifically binds to mesothelin may be directly or indirectly labeled. An indirect label includes a secondary antibody comprising a detectable label, wherein the secondary antibody binds to an antibody that specifically binds to mesothelin. Another indirect label includes biotin, wherein an antibody that specifically binds to biotinylated mesothelin can be detected using avidin or streptavidin containing a detectable label.

A suitable detectable label includes any composition detectable by spectroscopic, photochemical, biochemical, immunochemical, electrical, optical or chemical means. A suitable labels includes, but are not limited to, magnetic beads, fluorescent dyes (e.g., fluorescein isothiocyanate, Texas red, rhodamine, green fluorescent protein, red fluorescent protein, yellow fluorescent protein, etc.), radioactive labels (e.g., For example, ³H, ¹²⁵I, ³⁵S, ⁴C or ³²P), enzymes (e.g., mustard radish peroxidase, alkaline phosphatase, luciferase and the ones commonly used for enzyme-linked immunosorbent assay (ELISA)) and colorimetric labels such as colloidal gold or tinted glass or plastic (e.g., polystyrene, polypropylene, latex, etc.) beads.

In addition, for diagnosis or monitoring, the antibody may be labeled with a fluorescent protein, and may contain a contrast agent or a radioisotope.

When the antibody that specifically binds to mesothelin of the present invention is used in a diagnostic kit, the antibody is immobilized on a support, and the support may be a microplate, microarray, chip, glass, bead or particle, or a membrane.

Hereinafter, preferred examples are presented to help the understanding of the present invention. However, the following examples are only provided for easier understanding of the present invention, and the contents of the present invention are not limited by the following examples.

### Example 1: Preparation and Selection of the Antibody that Specifically Bound to Mesothelin

To select the antibody specific for mesothelin peptide, a hybridoma producing the antibody that bound to mesothelin was prepared and the antibody was selected.

First, splenocytes were extracted by immunization with mesothelin protein (Acrobiosystems, cat #MSN-H5223), and hybridoma cells were prepared through cell fusion with mouse myeloma cells.

Mouse myeloma cells used for cell fusion cannot survive in HAT medium because they do not have HypoxanthineGuanidine-Phosphoribosyl-Transferase (HGPRT), but hybridomas can survive in HAT medium by fusing with splenocytes. Since only hybridomas can be grown using this, they were usually grown in HAT medium until hybridomas were established.

A limiting dilution method was used to select hybridomas producing antibodies binding to mesothelin among the proliferated hybridomas. First, it was made to be less than one cell per 96 well, and then, it was confirmed by ELISA whether the antibody obtained from clones proliferated from one cell bound to mesothelin, and clones that bound to mesothelin were selected. The above process was repeated three times to select hybridomas producing the antibody that bound to mesothelin. In this way, seven types of antibodies that bound to mesothelin were obtained.

The seven types of antibodies were named 3A8, 4G11, 5A9, 6G5, 7C3, 9E8 and 9E11, respectively, and their nucleotide and amino acid sequences were analyzed. Sequence information for a heavy chain variable region and a light chain variable region of each antibody according to the sequencing result was shown in Tables 1 to 7 below, and the underlined parts in Tables 1 to 7 are complementary determining regions (CDRs).

TABLE 1
Sequence information of 3A8 antibody

| 3A8 | sequence information | SEQ ID NO: |
|---|---|---|
| Heavy chain | GYSFTGYT | SEQ ID NO: 1 |
| variable region | | |
| CDR1 | | |
| Heavy chain | INPYNGGT | SEQ ID NO: 2 |
| variable region | | |
| CDR2 | | |
| Heavy chain | ARVGGSSWYFDV | SEQ ID NO: 3 |
| variable region | | |
| CDR3 | | |
| Light chain variable | QSLLYSSNQKNY | SEQ ID NO: 4 |
| region CDR1 | | |
| Light chain variable | WAS | |
| region CDR2 | | |
| Light chain variable | QQYYSYPTWT | SEQ ID NO: 6 |
| region CDR3 | | |
| Amino acid | EVQLQQSGPELVKPGASMKISCKASGYSFTGYTMNW | SEQ ID NO: 7 |
| sequence of Heavy | VKQSHGKNLEWIGLINPYNGGTSYNQKFKGKATLTV | |
| chain variable | DKSSSTAYMELLSLTSEDSAVYYCARVGGSSWYFDV | |
| region | WGAGTTVTVSS | |
| Amino acid | DIVMTQSPSSLAVSVGEKVIMSCKSSQSLLYSSNQKN | SEQ ID NO: 8 |
| sequence of Light | YLAWYQQKPGQSPKLLIYWASTRESGVPDRFTGSGS | |
| chain variable | GTDFTLTISSVKAEDLAVYYCQQYYSYPTWTFGGGTK | |
| region | LEIK | |
| Nucleotide | gaggtccagctgcaacagtctggacctgagctggtgaagcctgga | SEQ ID NO: 9 |
| sequence of Heavy | gcttcaatgaagatatcctgcaaggcttctggttactcattcactggc | |
| chain variable | tacaccatgaactgggtgaagcagagccatggaaagaaccttgag | |
| region | tggattggacttattaatccttacaatggtggtactagctacaacca | |
| | gaagttcaagggcaaggccacattaactgtagacaagtcatccag | |
| | cacagcctacatggagctcctcagtctgacatctgaggactctgca | |
| | gtctattactgtgcaagggtgggcggtagtagctggtacttcgatgt | |
| | ctggggcgcagggaccacggtcaccgtctcctca | |
| Nucleotide | gacattgtgatgacccagtctccatcctccctagctgtgtcagttgga | SEQ ID NO: 10 |
| sequence of Light | gagaaggttattatgagctgcaagtccagtcagagccttttatatag | |
| chain variable | tagcaatcaaaagaactacttggcctggtaccagcagaaaccagg | |
| region | gcagtctcctaaactgctgatttactgggcatccactagggaatctg | |
| | gggtccctgatcgcttcacaggcagtggatctgggacagatttcact | |
| | ctcaccatcagcagtgtgaaggctgaagacctggcagtttattactg | |
| | tcagcaatattatagctatcctacgtggacgttcggtggaggcacca | |
| | agctggaaatcaaa | |

TABLE 2
Sequence information of 4G11 antibody

| 4G11 | sequence information | SEQ ID NO: |
|---|---|---|
| Heavy chain | GYSFTGYY | SEQ ID NO: 11 |
| variable region | | |
| CDR1 | | |
| Heavy chain | ISCYNGAT | SEQ ID NO: 12 |
| variable region | | |
| CDR2 | | |
| Heavy chain | ARWDRDWFAY | SEQ ID NO: 13 |
| variable region | | |
| CDR3 | | |
| Light chain variable | QDVGIA | SEQ ID NO: 14 |
| region CDR1 | | |
| Light chain variable | WAS | |
| region CDR2 | | |
| Light chain variable | QQYSSYPFT | SEQ ID NO: 16 |
| region CDR3 | | |
| Amino acid | EVQLQQSGPELVKTGDSVKISCKASGYSFTGYYMHW | SEQ ID NO: 17 |
| sequence of Heavy | VKQSHGKSLEWIGYISCYNGATSYSQKFKGKATFTVD | |
| chain variable | TSSSTAYMQFNSLTSEDSAVYYCARWDRDWFAYWG | |
| region | QGTLVTVSA | |
| Amino acid | DIVMTQSHKFMSTSVGDRVSITCKASQDVGIAVAWY | SEQ ID NO: 18 |
| sequence of Light | QQKPGQSPKLLIYWASTRHTGVPDRFTGSGSGTDFTL | |
| chain variable | TISNVQSEDLADYFCQQYSSYPFTFGSGTKLEIK | |
| region | | |
| Nucleotide | taggtccagctgcaacagtctggacctgagctagtgaagactgggg | SEQ ID NO: 19 |
| sequence of Heavy | attcagtgaagatatcctgcaaggcttctggttactcattcactggtt | |
| chain variable | actacatgcactgggtcaagcagagccatggaaagagccttgagt | |
| region | ggattggatatattagttgttacaatggtgctactagctacagccag | |
| | aagttcaagggcaaggccacatttactgtagacacatcctccagca | |
| | cagcctacatgcagttcaacagcctgacatctgaagactctgcggt | |
| | ctattactgtgcaagatgggacagggactggtttgcttactggggcc | |
| | aagggactctggtcactgtctctgca | |
| Nucleotide | tacattgtgatgacccagtctcacaaattcatgtccacatcagtggg | SEQ ID NO: 20 |
| sequence of Light | agacagggtcagcatcacctgcaaggccagtcaggatgtgggtatt | |
| chain variable | gctgtagcctggtatcaacagaaaccagggcaatctcctaaactac | |
| region | tgatttactgggcatccacccggcacactggagtccctgatcgcttc | |
| | acaggcagtggatctgggacagatttcactctcaccattagcaatgt | |
| | gcagtctgaagacttggcagattatttctgtcagcaatatagcagct | |
| | atccattcacgttcggctcggggacaaagttggaaataaaa | |

TABLE 3
Sequence information of 5A9 antibody

| 5A9 | sequence information | SEQ ID NO: |
|---|---|---|
| Heavy chain | GFSITSSSYC | SEQ ID NO: 21 |
| variable region | | |
| CDR1 | | |
| Heavy chain | ICYEGSI | SEQ ID NO: 22 |
| variable region | | |
| CDR2 | | |
| Heavy chain | SRENRLLKDAMDY | SEQ ID NO: 23 |
| variable region | | |
| CDR3 | | |
| Light chain variable | QSLLSSRTRKNY | SEQ ID NO: 24 |
| region CDR1 | | |
| Light chain variable | WAS | |
| region CDR2 | | |
| Light chain variable | KQSYNLRT | SEQ ID NO: 26 |
| region CDR3 | | |
| Amino acid | EVQLEESGPAVIKPSQSLSLTCIVSGFSITSSSYCWHWI | SEQ ID NO: 27 |
| sequence of Heavy | RQPPGKGLEWMGRICYEGSIYYSPSIKSRFTISRDTSLN | |
| chain variable | KFFIQLSSVTNEDTAMYYCSRENRLLKDAMDYWGQ | |
| region | GTSVTVSS | |
| Amino acid | DIVMTQSPSSLAVSAGEKVTMSCKSSQSLLSSRTRKN | SEQ ID NO: 28 |
| sequence of Light | YLAWYQQKPGQSPKLLIYWASTRESGVPDRFTGSGS | |
| chain variable | GTDFTLTISSVQAEDLAVYYCKQSYNLRTFGGGTKLEI | |
| region | K | |
| Nucleotide | caggtgcagctggaggagtctggacctgctgtcatcaagccatcac | SEQ ID NO: 29 |
| sequence of Heavy | agtcactgtctctcacctgcatagtctctggattctccatcacaagta | |
| chain variable | gtagttattgctggcactggatccgccagcccccaggaaaggggtt | |
| region | agagtggatggggcgcatatgttatgaaggttcaatatactatagtc | |
| | catccatcaaaagccgcttcaccatctccagagacacatctctgaa | |
| | caaattctttatccagctgagctctgtgacaaatgaggacacagcc | |
| | atgtactactgttccagggaaaaccgcctactgaaggacgctatgg | |
| | actactggggtcaaggaacctcagtcaccgtctcctca | |
| Nucleotide | aacattgtgatgacccagtctccatcctccctggctgtgtcagcagg | SEQ ID NO: 30 |
| sequence of Light | agagaaggtcactatgagctgcaaatccagtcagagtctgctcagc | |
| chain variable | agtagaacccgaaagaactacttggcttggtaccagcagaaacca | |
| region | gggcagtctcctaaactgctgatctactgggcatccactagggaatc | |
| | tggggtccctgatcgcttcacaggcagtggatctgggacagatttca | |
| | ctctcaccatcagcagtgtgcaggctgaagacctggcagtttattac | |
| | tgcaaacaatcttataatcttcggacgttcggtggaggcaccaagct | |
| | ggaaatcaaa | |

TABLE 4
Sequence information of 6G5 antibody

| 6G5 | sequence information | SEQ ID NO: |
|---|---|---|
| Heavy chain | GYSFTGYT | SEQ ID NO: 31 |
| variable region | | |
| CDR1 | | |
| Heavy chain | INPYNGGT | SEQ ID NO: 32 |
| variable region | | |
| CDR2 | | |
| Heavy chain | ARVGGSSWYFDV | SEQ ID NO: 33 |
| variable region | | |
| CDR3 | | |
| Light chain variable | QSLLYSSNQKNY | SEQ ID NO: 34 |
| region CDR1 | | |
| Light chain variable | WAS | |
| region CDR2 | | |
| Light chain variable | QQYYTYPTWT | SEQ ID NO: 36 |
| region CDR3 | | |
| Amino acid | EVQLQQSGPELVKPGASMKISCKASGYSFTGYTMNW | SEQ ID NO: 37 |
| sequence of Heavy | VKQSHGKNLEWIGLINPYNGGTSYNQKFKGKATLTV | |
| chain variable | DKSSSTAYMELLSLTSEDSAVYYCARVGGSSWYFDV | |
| region | WGAGTTVTVSS | |
| Amino acid | DIVMTQSPSSLAVSVGEKVTMSCKSSQSLLYSSNQKN | SEQ ID NO: 38 |
| sequence of Light | YLAWYQQKPGQSPKLLIYWASTRESGVPDRFTGSGS | |
| chain variable | GTDFTLTISSVKAEDLAVYYCQQYYTYPTWTFGGGTK | |
| region | LEIK | |
| Nucleotide | aaggtccagctgcaacagtctggacctgagctggtgaagcctgga | SEQ ID NO: 39 |
| sequence of Heavy | gcttcaatgaagatatcctgcaaggcttctggttactcattcactggc | |
| chain variable | tacaccatgaactgggtgaagcagagccatggaaagaaccttgag | |
| region | tggattggacttattaatccttacaatggtggtactagttacaacca | |
| | gaagttcaagggcaaggccacattaactgtagacaagtcatccag | |
| | cacagcctacatggagctcctcagtctgacatctgaggactctgca | |
| | gtctattactgtgcaagggtgggcggtagtagctggtacttcgatgt | |
| | ctggggcgcagggaccacggtcaccgtctcctca | |
| Nucleotide | tacattgtgatgacccagtctccatcctccctagctgtgtcagttgga | SEQ ID NO: 40 |
| sequence of Light | gagaaggttactatgagctgcaagtccagtcagagccttttatatag | |
| chain variable | tagcaatcaaaagaactacttggcctggtaccagcagaaaccagg | |
| region | gcagtctcctaaactgctgatttactgggcatccactagggaatctg | |
| | gggtccctgatcgcttcacaggcagtggatctgggacagatttcact | |
| | ctcaccatcagcagtgtgaaggctgaagacctggcagtttattactg | |
| | tcagcaatattatacctatcctacgtggacgttcggtggaggcacca | |
| | agctggaaatcaaa | |

TABLE 5
Sequence information of 7C3 antibody

| 7C3 | sequence information | SEQ ID NO: |
|---|---|---|
| Heavy chain | GYTFSAYW | SEQ ID NO: 41 |
| variable region | | |
| CDR1 | | |
| Heavy chain | ILPGSGST | SEQ ID NO: 42 |
| variable region | | |
| CDR2 | | |
| Heavy chain | ARGDYYAMDY | SEQ ID NO: 43 |
| variable region | | |
| CDR3 | | |
| Light chain variable | QSLLYSNGKTY | SEQ ID NO: 44 |
| region CDR1 | | |
| Light chain variable | LVS | |
| region CDR2 | | |
| Light chain variable | VQGTHFPFT | SEQ ID NO: 46 |
| region CDR3 | | |
| Amino acid | EVQLQQSGAELMRPGASVKISCKATGYTFSAYWIEW | SEQ ID NO: 47 |
| sequence of Heavy | VKQRPGHGLEWIGEILPGSGSTKYNEKFKGKATFTAD | |
| chain variable | TSSNTAYMQLSSLTSEDSAVYYCARGDYYAMDYWG | |
| region | QGTSVTVSS | |
| Amino acid | DVLMTQTPLTLSVTIGQPASISCKSSQSLLYSNGKTYL | SEQ ID NO: 48 |
| sequence of Light | NWLLQRPGQSPKRLIYLVSKLDSGVPDRFTGSGSGTD | |
| chain variable | FTLKISRVEAEDLGVYYCVQGTHFPFTFGSGTKLEIK | |
| region | | |
| Nucleotide | taggtccagctgcaacagtctggagctgagctgatgaggcctgggg | SEQ ID NO: 49 |
| sequence of Heavy | cctcagtgaagatatcctgcaaggctactggctacacattcagtgcc | |
| chain variable | tactggatagagtgggtaaagcagaggcctggacatggccttgagt | |
| region | ggattggagagattttacctggaagtggtagtactaaatacaatga | |
| | gaagttcaagggcaaggccacattcactgcagatacatcctccaac | |
| | acagcctacatgcaactcagcagcctgacatctgaggactctgccg | |
| | tctattactgtgcaagaggggattactatgctatggactactggggt | |
| | caaggaacctcagtcaccgtctcctca | |
| Nucleotide | aatgttttgatgacccaaactccactcactttgtcggttaccattgga | SEQ ID NO: 50 |
| sequence of Light | caaccagcctctatctcttgcaagtcaagtcagagcctcttatatag | |
| chain variable | taatggaaaaacctatttgaattggttattacagaggccaggccag | |
| region | tctccaaagcgcctaatctatctggtgtctaaactggactctggagt | |
| | ccctgacaggttcactggcagtggatcaggaacagattttacactg | |
| | aaaatcagcagagtggaggctgaggatttgggagtttattactgcg | |
| | tgcaaggtacacattttccattcacgttcggctcggggacaaagttg | |
| | gaaataaaa | |

TABLE 6
Sequence information of 9E8 antibody

| 9E8 | sequence information | SEQ ID NO: |
|---|---|---|
| Heavy chain | GYSFTGYT | SEQ ID NO: 51 |
| variable region | | |
| CDR1 | | |
| Heavy chain | INPYNGGT | SEQ ID NO: 52 |
| variable region | | |
| CDR2 | | |
| Heavy chain | ARVGGSSWYFDV | SEQ ID NO: 53 |
| variable region | | |
| CDR3 | | |
| Light chain variable | QSLLYSSNQKNY | SEQ ID NO: 54 |
| region CDR1 | | |
| Light chain variable | WAS | |
| region CDR2 | | |
| Light chain variable | QQYYSYPTWT | SEQ ID NO: 56 |
| region CDR3 | | |
| Amino acid | EVQLQQSGPELVKPGASMKISCKASGYSFTGYTMNW | SEQ ID NO: 57 |
| sequence of Heavy | VKQSHGKNLEWIGLINPYNGGTSYNQKFKGKATLTV | |
| chain variable | DKSSSTAYMELLSLTSEDSAVYYCARVGGSSWYFDV | |
| region | WGAGTTVTVSS | |
| Amino acid | DIVMTQSPSSLAVSVGEKVIMSCKSSQSLLYSSNQKN | SEQ ID NO: 58 |
| sequence of Light | YLAWYQQKPGQSPKLLIYWASTRESGVPDRFTGSGS | |
| chain variable | GTDFTLTISSVKAEDLAVYYCQQYYSYPTWTFGGGTK | |
| region | LEIK | |
| Nucleotide | gaggtccagctgcaacagtctggacctgagctggtgaagcctgga | SEQ ID NO: 59 |
| sequence of Heavy | gcttcaatgaagatatcctgcaaggcttctggttactcattcactggc | |
| chain variable | tacaccatgaactgggtgaagcagagccatggaaagaaccttgag | |
| region | tggattggacttattaatccttacaatggtggtactagctacaacca | |
| | gaagttcaagggcaaggccacattaactgtagacaagtcatccag | |
| | cacagcctacatggagctcctcagtctgacatctgaggactctgca | |
| | gtctattactgtgcaagggtgggcggtagtagctggtacttcgatgt | |
| | ctggggcgcagggaccacggtcaccgtctcctca | |
| Nucleotide | gacattgtgatgacccagtctccatcctccctagctgtgtcagttgga | SEQ ID NO: 60 |
| sequence of Light | gagaaggttattatgagctgcaagtccagtcagagccttttatatag | |
| chain variable | tagcaatcaaaagaactacttggcctggtaccagcagaaaccagg | |
| region | gcagtctcctaaactgctgatttactgggcatccactagggaatctg | |
| | gggtccctgatcgcttcacaggcagtggatctgggacagatttcact | |
| | ctcaccatcagcagtgtgaaggctgaagacctggcagtttattactg | |
| | tcagcaatattatagctatcctacgtggacgttcggtggaggcacca | |
| | agctggaaatcaaa | |

TABLE 7
Sequence information of 9E11 antibody

| 9E11 | sequence information | SEQ ID NO: |
|---|---|---|
| Heavy chain | GYSITSDYA | SEQ ID NO: 61 |
| variable region | | |
| CDR1 | | |
| Heavy chain | ISYSGST | SEQ ID NO: 62 |
| variable region | | |
| CDR2 | | |
| Heavy chain | ARGAAGFAY | SEQ ID NO: 63 |
| variable region | | |
| CDR3 | | |
| Light chain variable | QTIGTW | SEQ ID NO: 64 |
| region CDR1 | | |
| Light chain variable | AAT | |
| region CDR2 | | |
| Light chain variable | QQLYSTPWT | SEQ ID NO: 66 |
| region CDR3 | | |
| Amino acid | QVQLKESGPGLVKPSQSLSLTCTVTGYSITSDYAWNW | SEQ ID NO: 67 |
| sequence of Heavy | IRQFPGNKLEWMGYISYSGSTRYNPSLKSRISITRDTSK | |
| chain variable | NQFFLQLNSVTTEDTATYYCARGAAGFAYWGQGTLV | |
| region | TVSA | |
| Amino acid | DIVMTQSPASQSASLGESVTITCLASQTIGTWLAWYQ | SEQ ID NO: 68 |
| sequence of Light | QKPGKSPQLLIYAATSLADGVPSRFSGSGSGTEFSFKIS | |
| chain variable | SLQAEDFVSYYCQQLYSTPWTFGGGTKLEIK | |
| region | | |
| Nucleotide | gaggtgcagctgaaggagtcgggacctggcctggtgaaaccttcgc | SEQ ID NO: 69 |
| sequence of Heavy | agtctctgtccctcacctgcactgtcactggctactcaatcaccagtg | |
| chain variable | attatgcctggaactggatccggcagtttccaggaaacaaactgga | |
| region | gtggatgggctacataagctacagtggaagcactaggtacaaccc | |
| | atctctcaaaagtcgaatctctatcactcgagacacatccaagaac | |
| | cagttcttcctgcagttgaattctgtgactactgaggacacagccac | |
| | atattactgtgcaagaggggctgcgggctttgcttactggggccaa | |
| | gggactctggtcactgtctctgca | |
| Nucleotide | tacattgtgatgacccagtctcctgcctcccagtctgcatctctggga | SEQ ID NO: 70 |
| sequence of Light | gaaagtgtcaccatcacatgcctggcaagtcagaccattggtacat | |
| chain variable | ggttagcatggtatcagcagaaaccagggaaatctcctcagctcct | |
| region | gatttatgctgcaaccagcttggcagatggggtcccatcaaggttca | |
| | gtggtagtggatctggcacagaattttctttcaagatcagcagccta | |
| | caggctgaagattttgtaagttattactgtcaacaactttacagtact | |
| | ccgtggacgttcggtggaggcaccaagctggaaatcaaa | |

### Example 2: Confirmation of Specificity of the Selected Antibody for Mesothelin-ELISA Analysis

In the present invention, in order to confirm the specificity for mesothelin of 3A8, 4G11, 5A9, 6G5, 7C3, 9E8 and 9E11 antibodies established in Example 1, ELISA analysis was performed.

First, in order to encode the mesothelin peptide, mesothelin peptide was dispensed into a 96-well plate at an amount of 100 ng/well and reacted overnight at 4° C. Then, after treatment with 1×PBST containing 3% BSA, it was blocked for 30 minutes at room temperature. 100 μl of hybridoma cell culture of each clone producing 3A8, 4G11, 5A9, 6G5, 7C3, 9E8 or 9E11 antibody was treated in each well, reacted at room temperature for 2 hours, and then washed three times with 1×PBST. The secondary antibody (anti-HRP, 1:10,000) was treated and reacted at room temperature for 30 minutes, washed three times with 1×PBST, and then treated with TMB for color development and reacted at room temperature for 5 minutes. Finally, the reaction was terminated by treatment with a stop solution of 1N H₂SO₄, and then the absorbance was measured at 450 nm.

TABLE 8
ELISA experimental conditions

| ELISA reader | Infinite F50 |
|---|---|
| Measurement Filter | 450 nm |
| Measurement Mode | Single Point Photo |
| Antigen Coating | 100 ng/well |
| 2nd Antibody (Anti-mlgG-HRP) | 1:10,000 dilution |
| Substrate | TMB |

TABLE 9
ELISA experiment results

| antibody type | OD ₄₅₀ measurements |
|---|---|
| 3A8 | 1.270 |
| 4G11 | 1.352 |
| 5A9 | 1.487 |
| 6G5 | 1.424 |
| 7C3 | 1.518 |
| 9E8 | 0.758 |
| 9E11 | 1.640 |

As a result, as shown in Table 9, it was confirmed that all antibodies selected in the present invention specifically bound to mesothelin.

### Example 3: Confirmation of Specificity of the Selected Antibody to Mesothelin—Flow Cytometer

In the present invention, in order to confirm the specificity for mesothelin of 3A8, 4G11, 5A9, 6G5, 7C3, 9E8 and 9E11 antibodies established in Example 1, flow cytometry was performed.

First, NCI-H2052 (4×10⁵), a mesothelioma cell line overexpressing mesothelin, was reacted with 3A8, 4G11, 5A9, 6G5, 7C3, 9E8, and 9E11 antibodies (1 μg each), respectively, for 30 min, then the surface was stained with secondary antibodies, and then measured by flow cytometry.

Mesothelin antibody (APC anti-human Mesothelin; R&D Systems, cat #FAB32652A, 5 μl) was used as a positive control, and PE-conjugated goat anti-mouse IgG antibody (PE-conjugated goat anti-mouse IgG; Biolegend Inc., cat #405307, USA, 5 μl) was used as a secondary antibody.

TABLE 10
Flow cytometry results

| antibody type | Count | Median | Mean |
|---|---|---|---|
| 3A8 | 10094 | 704 | 861 |
| 4G11 | 10132 | 1116 | 1256 |
| 5A9 | 10120 | 58.5 | 63.2 |
| 6G5 | 10056 | 560 | 705 |
| 7C3 | 10111 | 444 | 528 |
| 9E8 | 10090 | 56.2 | 77.7 |
| 9E11 | 10123 | 69.1 | 81.4 |
| Positive control | 10101 | 53.4 | 57.8 |
| Secondary antibody | 10120 | 55.1 | 62.7 |
| treatment only | | | |
| (2nd Ab alone) | | | |
| No treatment (none) | 10080 | 13.7 | 14.1 |

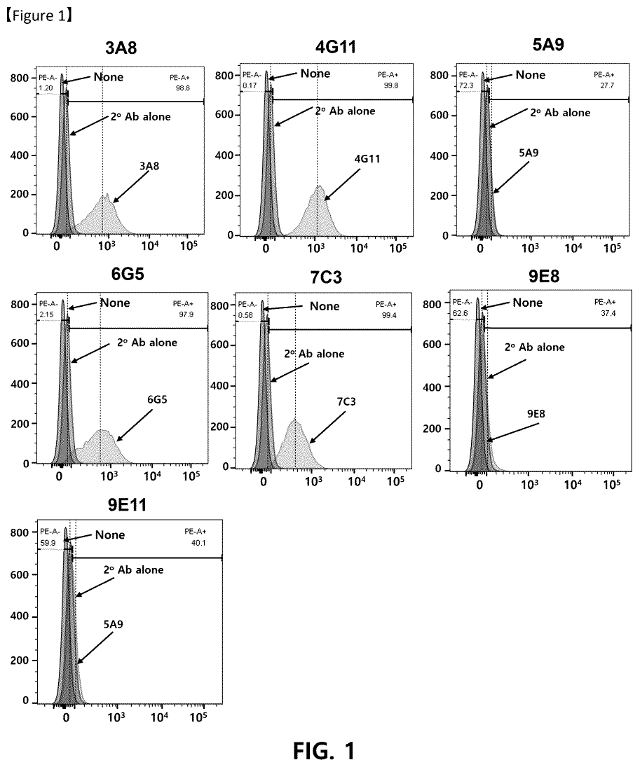

As a result, as shown in FIG. 1 and Table 10, it was confirmed that all of 3A8, 4G11, 5A9, 6G5, 7C3, 9E8, and 9E11 antibodies specifically bound to cells expressing mesothelin.

### Example 4: Vector Construction Expressing Chimeric Antigen Receptor Targeting Mesothelin (MSLN-CAR)

In the present invention, a lentiviral vector expressing a chimeric antigen receptor targeting mesothelin (MSLN-CAR lentivirus) was prepared using 3A8, 4G11, 6G5 and 7C3 antibodies prepared in Example 2 above.

As shown in the schematic of FIG. 3, DNA of CAR composed of:
EF1α promoter (SEQ ID NO: 87);
a polynucleotide encoding a signal peptide (SEQ ID NO: 88);
a polynucleotide encoding the mesothelin-binding domain (SEQ ID NO: 74, SEQ ID NO: 76, SEQ ID NO: 80, or SEQ ID NO: 82);
a polynucleotide encoding the CD8 hinge region (SEQ ID NO: 89);
a polynucleotide encoding a transmembrane domain (SEQ ID NO: 90);
a polynucleotide encoding 4-1BB (co-stimulatory domain) (SEQ ID NO: 91);
a polynucleotide encoding CD3ζ (intracellular signaling domain) (SEQ ID NO: 92); and
a polynucleotide encoding WPRE (SEQ ID NO: 93);
was synthesized in vitro and inserted into a third-generation lentiviral vector.

Lentiviral vector DNA (0.5 μg) was transferred to HEK293FT cells (5×10⁵ cells/500 μl), and 293HEK cells expressing the MSLN-CAR gene were constructed. Lipofectamine 3000 transfection kit (Invitrogen, cat #L3000-015) was used to transfer the gene to 293HEK cells, and cultured in Opti-MEM (gibco, cat #51985-034) medium for 4 hours.

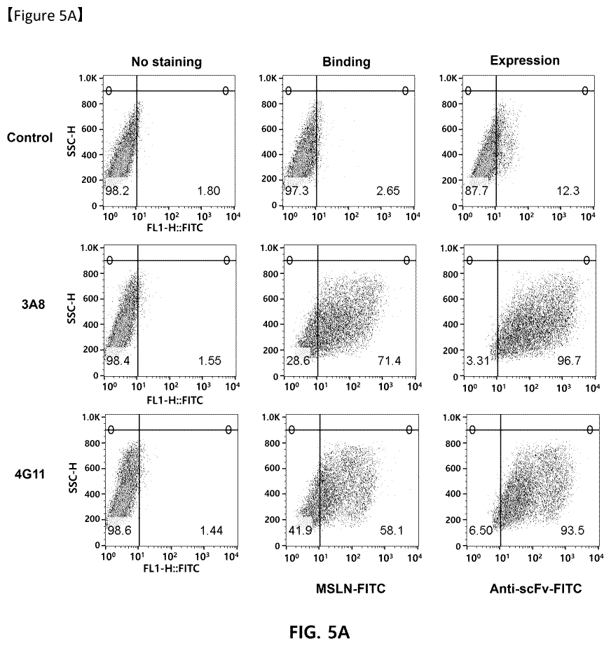

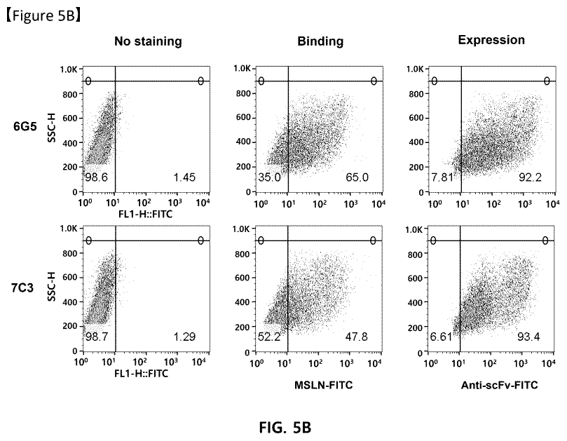

As a result of confirming through flow cytometry method whether MSLN-specific CAR is normally expressed and binds to MSLN peptide in HEK293FT transformed with lentiviral vector DNA (FIG. 4), as shown in FIGS. 5A and 5B, MSLN-CAR was confirmed to be expressed normally and bind to MSLN peptide.

### Example 5: Preparation of MSLN-CAR-T Cells

In the present invention, anti-MSLN antibody (3A8, 4G11, 6G5 or 7C3 antibody) based MSLN-CAR-T cells were prepared by transducing the MSLN-CAR lentiviral vector prepared in Example 4 above into T cells, respectively.

Specifically, peripheral blood mononuclear cells (PBMCs) were isolated from blood, and then T cells were activated using T cell activation beads (T cell activation beads; Miltenyl Biotec, cat #130-091-441), as shown in the schematic shown in FIGS. 6 and 7. MSLN-CAR-T cells were prepared by transfecting the activated T cells with the MSLN-CAR lentivirus prepared in Example 4 above.

The ability of MSLN-CAR-T cells to bind mesothelin peptide was confirmed by flow cytometry. The MSLN-CAR-T cells prepared above were classified as CD3-, CD4-, or CD8-activated MSLN-CAR-T cells using anti-CD3, anti-CD4, or anti-CD8 antibodies, respectively, and then reacted with mesothelin (FITC-MSLN) peptide, and the fluorescence intensity was measured using a flow cytometer.

As a result, it was found that both CD3-, CD4-, or CD8-activated MSLN-CAR-T cells bound to the mesothelin peptide, as shown in FIGS. 8A to 8D.

### Example 6: Confirmation of MSLN-CAR-T Cell Activation in Mesothelin-Expressing Cells

To confirm that the MSLN-CAR-T cells prepared in Example 5 are activated in mesothelin-expressing cells, the degree of IFNγ expression by MSLN-CAR-T cells in the presence of target cells was confirmed.

The target cells were H28 cells (mesothelioma cell line), which does not express mesothelin, and H2052 cells (mesothelioma cell line), which overexpresses mesothelin, and the target cells and MSLN-CAR-T cells were reacted for a certain period of time in a ratio of 1:2, 1:1, and 1:0.5, and then stained with surface and intra antibodies and measured by flow cytometer (INF-r, CD3, CD4, CD8 staining).

As a result, as shown in FIG. 9A to FIG. 9D, T cells were not activated in H28 cells that did not express mesothelin, while T cells were activated in the presence of H2052 cells overexpressing mesothelin, resulting in increased IFNγ expression.

### Example 7: Confirmation of the Killing Effect of MSLN-CAR-T Cells on Mesothelin-Expressing Cells

This example confirmed the killing effect of target cells by MSLN-CAR-T cells based on anti-mesothelin antibody prepared in Example 5 above.

The target cells were H28 cells that do not express mesothelin and H2052 cells that overexpress mesothelin, and the target cells and MSLN-CAR-T cells were mixed in a ratio of 1:20, 1:10, 1:4, 1:2, and 1:1, respectively, and incubated for 8 hours, and then luminescence (CytoTox-Glo Cytotoxicity Assay, Promega, cat. NO G9291) was measured. The measured values were used to calculate the degree of cell death using the following equation.

$$\% Cytotoxicity = [ ( Experimental Spontaneous - Target Spontaneous ) / \\ ( Target Maximum - Target Spontaneous ) ] \times 100 \qquad [ Equation 1 ]$$

Experimental: Luminescence value derived from the medium of the target cell and CAR-T cell combination culture.
Effector Spontaneous: Luminescence value derived from the medium of CAR-T cells alone
Target Spontaneous: Luminescence value derived from the medium of target cells only
Target Maximum: Luminescence value derived from 100% lysis of target cells (using Lysis Reagent).

As a result, as shown in FIG. 10, it was confirmed that MSLN-CAR-T cells based on anti-mesothelin antibodies (3A8 and 4G11) specifically killed H2052 cells overexpressing mesothelin.

Thus, it was confirmed that the seven antibodies (3A8, 4G11, 5A9, 6G5, 7C3, 9E8, and 9E11) specific for mesothelin selected in the present invention specifically bound to mesothelin antigen and enable the preparation of chimeric antigen receptor (CAR) and CAR-T cells (MSLN-CAR-T cells) targeting mesothelin. Furthermore, we have confirmed that the MSLN-CAR-T cells produced by the present invention activated MSLN-CAR-T cells in the presence of mesothelin antigen and effectively killed cells overexpressing mesothelin, so that the antibodies specific for mesothelin and the chimeric antigen receptors and CAR-T cells produced using them can be applied to the prevention or treatment of cancer or tumors in which mesothelin is overexpressed.

1. An antibody specifically binding to mesothelin or a fragment thereof, comprising:
(1) a heavy chain variable region comprising a CDR1 region represented by the amino acids of SEQ ID NO: 1, a CDR2 region represented by the amino acids of SEQ ID NO: 2, and a CDR3 region represented by the amino acids of SEQ ID NO: 3, and a light chain variable region comprising a CDR1 region including a CDR1 region represented by the amino acids of SEQ ID NO: 4, a CDR2 region having amino acids WAS, and a CDR3 region represented by the amino acids of SEQ ID NO: 6;
(2) a heavy chain variable region comprising a CDR1 region represented by the amino acids of SEQ ID NO: 11, a CDR2 region represented by the amino acids of SEQ ID NO: 12, and a CDR3 region represented by the amino acids of SEQ ID NO: 13, and a light chain variable region comprising a CDR1 region including a CDR1 region represented by the amino acids of SEQ ID NO: 14, a CDR2 region having amino acids WAS, and a CDR3 region represented by the amino acids of SEQ ID NO: 16;
(3) a heavy chain variable region comprising a CDR1 region represented by the amino acids of SEQ ID NO: 21, a CDR2 region represented by the amino acids of SEQ ID NO: 22, and a CDR3 region represented by the amino acids of SEQ ID NO: 23, and a light chain variable region comprising a CDR1 region including a CDR1 region represented by the amino acids of SEQ ID NO: 24, a CDR2 region having amino acids WAS, and a CDR3 region represented by the amino acids of SEQ ID NO: 26;
(4) a heavy chain variable region comprising a CDR1 region represented by the amino acids of SEQ ID NO: 31, a CDR2 region represented by the amino acids of SEQ ID NO: 32, and a CDR3 region represented by the amino acids of SEQ ID NO: 33, and a light chain variable region comprising a CDR1 region including a CDR1 region represented by the amino acids of SEQ ID NO: 34, a CDR2 region having amino acids WAS, and a CDR3 region represented by the amino acids of SEQ ID NO: 36;
(5) a heavy chain variable region comprising a CDR1 region represented by the amino acids of SEQ ID NO: 41, a CDR2 region represented by the amino acids of SEQ ID NO: 42, and a CDR3 region represented by the amino acids of SEQ ID NO: 43, and a light chain variable region comprising a CDR1 region including a CDR1 region represented by the amino acids of SEQ ID NO: 44, a CDR2 region having amino acids LVS, and a CDR3 region represented by the amino acids of SEQ ID NO: 46;
(6) a heavy chain variable region comprising a CDR1 region represented by the amino acids of SEQ ID NO: 51, a CDR2 region represented by the amino acids of SEQ ID NO: 52, and a CDR3 region represented by the amino acids of SEQ ID NO: 53, and a light chain variable region comprising a CDR1 region including a CDR1 region represented by the amino acids of SEQ ID NO: 54, a CDR2 region having amino acids WAS, and a CDR3 region represented by the amino acids of SEQ ID NO: 56; or
(7) a heavy chain variable region including a CDR1 region represented by an amino acid of SEQ ID NO: 61, a CDR2 region represented by an amino acid of SEQ ID NO: 62 and a CDR3 region represented by an amino acid of SEQ ID NO: 63, and a light chain variable region including a CDR1 region represented by an amino acid of SEQ ID NO: 64, a CDR2 region having amino acids AAT and a CDR3 region represented by an amino acid of SEQ ID NO: 66.

2. The antibody specifically binding to mesothelin or a fragment thereof of claim 1,
wherein the (1) antibody comprises a heavy chain variable region represented by amino acids of SEQ ID NO: 7 and a light chain variable region represented by amino acids of SEQ ID NO: 8,
the (2) antibody comprises a heavy chain variable region represented by amino acids of SEQ ID NO: 17 and a light chain variable region represented by amino acids of SEQ ID NO: 18,
the (3) antibody comprises a heavy chain variable region represented by amino acids of SEQ ID NO: 27 and a light chain variable region represented by amino acids of SEQ ID NO: 28,
the (4) antibody comprises a heavy chain variable region represented by amino acids of SEQ ID NO: 37 and a light chain variable region represented by amino acids of SEQ ID NO: 38,
the (5) antibody comprises a heavy chain variable region represented by amino acids of SEQ ID NO: 47 and a light chain variable region represented by amino acids of SEQ ID NO: 48;
the (6) antibody comprises a heavy chain variable region represented by amino acids of SEQ ID NO: 57 and a light chain variable region represented by amino acids of SEQ ID NO: 58 or
the (7) antibody may comprise a heavy chain variable region represented by an amino acid of SEQ ID NO: 67 and a light chain variable region represented by an amino acid of SEQ ID NO: 68.

3. A polynucleotide encoding the antibody specifically binding to mesothelin or a fragment thereof of claim 1.

4. A vector comprising the polynucleotide encoding the antibody specifically binding to mesothelin or a fragment thereof of claim 1.

5. A recombinant cell producing the antibody or fragment thereof specifically binding to mesothelin or a fragment thereof transformed with the vector of claim 4.

6. A pharmaceutical composition comprising the antibody or fragment thereof that specifically binds to the mesothelin of claim 1 and a pharmaceutically acceptable carrier.

7. A method prevention or treatment of cancer or tumors in a subject comprising administering the pharmaceutical composition of claim 6 to the subject.

8. A composition for diagnosis or monitoring of a mesothelin-overexpressing cancer or tumor, comprising the antibody or fragment thereof that specifically binds to the mesothelin of claim 1.

9. A chimeric antigen receptor (CAR) targeting mesothelin comprising: a mesothelin-binding domain; a transmembrane domain; a costimulatory domain; and an intracellular signal transduction domain,
wherein the mesothelin-binding domain is an antibody specifically binding to mesothelin or a fragment thereof, comprising:
(1) a heavy chain variable region comprising a CDR1 region represented by the amino acids of SEQ ID NO: 1, a CDR2 region represented by the amino acids of SEQ ID NO: 2, and a CDR3 region represented by the amino acids of SEQ ID NO: 3, and a light chain variable region comprising a CDR1 region including a CDR1 region represented by the amino acids of SEQ ID NO: 4, a CDR2 region having amino acids WAS, and a CDR3 region represented by the amino acids of SEQ ID NO: 6;
(2) a heavy chain variable region comprising a CDR1 region represented by the amino acids of SEQ ID NO: 11, a CDR2 region represented by the amino acids of SEQ ID NO: 12, and a CDR3 region represented by the amino acids of SEQ ID NO: 13, and a light chain variable region comprising a CDR1 region including a CDR1 region represented by the amino acids of SEQ ID NO: 14, a CDR2 region having amino acids WAS, and a CDR3 region represented by the amino acids of SEQ ID NO: 16;
(3) a heavy chain variable region comprising a CDR1 region represented by the amino acids of SEQ ID NO: 21, a CDR2 region represented by the amino acids of SEQ ID NO: 22, and a CDR3 region represented by the amino acids of SEQ ID NO: 23, and a light chain variable region comprising a CDR1 region including a CDR1 region represented by the amino acids of SEQ ID NO: 24, a CDR2 region having amino acids WAS, and a CDR3 region represented by the amino acids of SEQ ID NO: 26;
(4) a heavy chain variable region comprising a CDR1 region represented by the amino acids of SEQ ID NO: 31, a CDR2 region represented by the amino acids of SEQ ID NO: 32, and a CDR3 region represented by the amino acids of SEQ ID NO: 33, and a light chain variable region comprising a CDR1 region including a CDR1 region represented by the amino acids of SEQ ID NO: 34, a CDR2 region having amino acids WAS, and a CDR3 region represented by the amino acids of SEQ ID NO: 36;
(5) a heavy chain variable region comprising a CDR1 region represented by the amino acids of SEQ ID NO: 41, a CDR2 region represented by the amino acids of SEQ ID NO: 42, and a CDR3 region represented by the amino acids of SEQ ID NO: 43, and a light chain variable region comprising a CDR1 region including a CDR1 region represented by the amino acids of SEQ ID NO: 44, a CDR2 region having amino acids LVS, and a CDR3 region represented by the amino acids of SEQ ID NO: 46;
(6) a heavy chain variable region comprising a CDR1 region represented by the amino acids of SEQ ID NO: 51, a CDR2 region represented by the amino acids of SEQ ID NO: 52, and a CDR3 region represented by the amino acids of SEQ ID NO: 53, and a light chain variable region comprising a CDR1 region including a CDR1 region represented by the amino acids of SEQ ID NO: 54, a CDR2 region having amino acids WAS, and a CDR3 region represented by the amino acids of SEQ ID NO: 56; or
(7) a heavy chain variable region including a CDR1 region represented by an amino acid of SEQ ID NO: 61, a CDR2 region represented by an amino acid of SEQ ID NO: 62 and a CDR3 region represented by an amino acid of SEQ ID NO: 63, and a light chain variable region including a CDR1 region represented by an amino acid of SEQ ID NO: 64, a CDR2 region having amino acids AAT and a CDR3 region represented by an amino acid of SEQ ID NO: 66.

10. The chimeric antigen receptor of claim 9, wherein the transmembrane domain is a protein selected from the group consisting of CD8α, CD4, CD28, CD137, CD80, CD86, CD152 and PD1,
the costimulatory domain is a protein selected from the group consisting of CD28, 4-1BB, OX-40 and ICOS, and
the intracellular signal transduction domain is CD3ζ.

11. The chimeric antigen receptor of claim 9, further comprising a hinge region between a C-terminus of the mesothelin-binding domain and an N-terminus of the transmembrane domain.

12. A polynucleotide encoding the chimeric antigen receptor of claim 9.

13. A vector comprising the polynucleotide encoding the chimeric antigen receptor of claim 9.

14. An immune effector cell comprising the polynucleotide encoding the chimeric antigen receptor targeting mesothelin of claim 9 or the vector containing the polynucleotide.

15. A pharmaceutical composition for use in preventing or treating a cancer or tumor expressing mesothelin, comprising the immune effector cell of claim 14.

16. A method of preventing or treating a cancer or tumor expressing mesothelin in a subject comprising administering the pharmaceutical composition of claim 15 to the subject.

17. The method of claim 16, wherein the cancer or tumor expressing mesothelin is selected from the group consisting of a squamous cell carcinoma, a small cell lung cancer, a non-small cell lung cancer, an adenocarcinoma of the lung, a squamous cell carcinoma of the lung, a mesothelioma, a peritoneal cancer, a hepatocellular carcinoma, a gastrointestinal cancer, a pancreatic cancer, a glioma, a cervical cancer, an ovarian cancer, a liver cancer, a bladder cancer, a hepatocellular carcinoma, a breast cancer, a colon cancer, a colorectal cancer, an endometrial carcinoma, a uterine carcinoma, a salivary gland carcinoma, a kidney cancer, a liver cancer, a prostate cancer, a vulvar cancer, a thyroid cancer, a liver carcinoma, a leukemia, a lymphoproliferative disorder, and a head and neck cancer.

18. The method of claim 7, wherein the cancer or tumor is selected from the group consisting of a squamous cell carcinoma, a small cell lung cancer, a non-small cell lung cancer, an adenocarcinoma of the lung, a squamous cell carcinoma of the lung, a mesothelioma, a peritoneal cancer, a hepatocellular carcinoma, a gastrointestinal cancer, a pancreatic cancer, a glioma, a cervical cancer, an ovarian cancer, a liver cancer, a bladder cancer, a hepatocellular carcinoma, a breast cancer, a colon cancer, a colorectal cancer, an endometrial carcinoma, a uterine carcinoma, a salivary gland carcinoma, a kidney cancer, a liver cancer, a prostate cancer, a vulvar cancer, a thyroid cancer, a liver carcinoma, a leukemia, a lymphoproliferative disorder, and a head and neck cancer.
